(19) United States
(12) Patent Application Publication
(10) Pub. No.: US 2012/0202840 A1
(43) Pub. Date: Aug. 9, 2012

(54) Use of Flubendazole and Vinca Alkaloids for Treatment of Hematological Diseases

(75) Inventors: Aaron David Schimmer, Thornhill (CA); Jiayi Hu, Toronto (CA); Paul Anthony Spangnuolo, Oakville (CA)

(73) Assignee: UNIVERSITY HEALTH NETWORK, Toronto (CA)

(21) Appl. No.: 13/500,162

(22) PCT Filed: Oct. 8, 2010
(86) PCT No.: PCT/CA2010/001627

§ 371 Date: Apr. 4, 2012

Related U.S. Application Data
(60) Provisional application No. 61/250,303, filed on Oct. 9, 2009.

(51) Int. Cl. A61K 31/475; A61K 31/4184; A61P 35/00; C07D 235/32; C07D 519/00

(52) U.S. Cl. 514/283; 548/308.7; 540/478; 514/395

(57) ABSTRACT

Provided are methods for treating a hematological malignancy comprising administering an effective amount of flubendazole alone or in combination with a vinca alkaloid. Also provided are compositions and kits comprising an effective amount of flubendazole and/or a vinca alkaloid for use in the methods of the disclosure.

### CROSS REFERENCE TO RELATED APPLICATIONS

This application is a PCT Patent Application, which claims the priority benefit of U.S. Provisional Patent Application No. 61/250,303, filed Oct. 9, 2009, which is incorporated herein by reference in its entirety.

### FIELD OF THE DISCLOSURE

The disclosure relates to methods and uses of flubendazole and vinca alkaloids for treating hematological malignancies and to compositions comprising flubendazole and vinca alkaloids.

### BACKGROUND OF THE DISCLOSURE

Flubendazole has been extensively evaluated in humans and animals for the treatment of intestinal parasites as well as for the treatment of systemic worm infections. In these studies, patients have received up to 50 mg/kg orally daily for 24 months without serious adverse effects.¹²⁻¹⁴ Healthy volunteers have also received single oral doses up to 2000 mg without toxicity.¹⁵ In sheep receiving a single flubendazole dose of 5 mg/kg intravenously, no toxicity was observed and the area under the curve (AUC) was 6.53 μg·h/mL (22 μM).¹⁶

Vinca alkaloids are currently used in the treatment of leukemia and myeloma, and neurotoxicity is a dose limiting toxicity of vincristine.

### SUMMARY OF THE DISCLOSURE

An aspect of the disclosure includes a method of treating a hematological malignancy in a subject in need thereof comprising administering to the subject an effective amount of flubendazole and/or a pharmaceutically acceptable solvate and/or prodrug thereof.

Another aspect of the disclosure includes a method of treating a hematological malignancy in a subject in need thereof comprising administering to the subject an effective amount of flubendazole and/or a pharmaceutically acceptable solvate and/or prodrug thereof, and an effective amount of a vinca alkaloid and/or a pharmaceutically acceptable salt, solvate and/or prodrug thereof.

In an embodiment, the vinca alkaloid is selected from vinblastine, vincristine, vindesine and/or vinolrebine and pharmaceutically acceptable salts, solvates and/or prodrugs thereof.

In another embodiment, the hematological malignancy is drug resistant to a vinca alkaloid and/or overexpresses P glycoprotein (Pgp).

In a further embodiment, the hematological malignancy is a leukemia, lymphoma or myeloma.

In yet another embodiment, the flubendazole and/or a pharmaceutically acceptable solvate and/or prodrug thereof and/or the vinca alkaloid and/or a pharmaceutically acceptable salt, solvate and/or prodrug thereof are comprised in a single oral dosage form or separate oral dosage forms.

In another embodiment, the flubendazole and/or a pharmaceutically acceptable solvate and/or prodrug thereof and/or the vinca alkaloid and/or a pharmaceutically acceptable salt, solvate and/or prodrug thereof are comprised in a single intravenous dosage form or separate intravenous dosage forms.

In another aspect, the disclosure includes use of an effective amount of flubendazole and/or a pharmaceutically acceptable solvate and/or prodrug thereof for treating a hematological malignancy.

In an embodiment, the disclosure includes use of an effective amount of flubendazole and/or a pharmaceutically acceptable solvate and/or prodrug thereof in combination with an effective amount of a vinca alkaloid and/or a pharmaceutically acceptable salt, solvate and/or prodrug thereof for treating a hematological malignancy.

A further aspect includes use of an effective amount of flubendazole and/or a pharmaceutically acceptable solvate and/or prodrug thereof for the manufacture of a medicament for treating a hematological malignancy.

In an embodiment, the disclosure includes use of an effective amount of flubendazole and/or a pharmaceutically acceptable solvate and/or prodrug thereof in combination with an effective amount of a vinca alkaloid and/or a pharmaceutically acceptable salt, solvate and/or prodrug thereof for the manufacture of a medicament for treating a hematological malignancy.

Another aspect of the disclosure includes a composition comprising an effective amount of flubendazole and/or a pharmaceutically acceptable solvate and/or prodrug thereof and a vinca alkaloid and/or a pharmaceutically acceptable salt, solvate and/or prodrug thereof.

In an embodiment, the disclosure includes a composition comprising an effective amount of flubendazole and/or a pharmaceutically acceptable solvate and/or prodrug thereof and optionally a vinca alkaloid and/or a pharmaceutically acceptable salt, solvate and/or prodrug thereof for treating a hematological malignancy.

A further aspect includes a kit comprising flubendazole and/or a pharmaceutically acceptable solvate and/or prodrug thereof and optionally a vinca alkaloid and/or a pharmaceutically acceptable salt, solvate and/or prodrug thereof for treating a hematological malignancy. In an embodiment, the kit comprises flubendazole and/or a pharmaceutically acceptable solvate and/or prodrug thereof and instructions for combination use with a vinca alkaloid and/or a pharmaceutically acceptable salt, solvate and/or prodrug thereof for treating a hematological malignancy.

Other features and advantages of the present disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

FIG. 1: Flubendazole induces cell death in malignant cell lines

(A) OCI-AML2 cells were treated for 72 h with increasing concentrations of benzimidazoles. After incubation, cell growth and viability was measured by the MTS assay. Data represent the mean percentage of viable cells ±SD from a representative experiment.

(B) Leukemia, lymphoma and myeloma cell lines were treated with increasing concentrations of flubendazole. Seventy two hours after incubation, cell growth and viability was measured by the MTS assay. Data represent the mean percentage of viable cells ±SD from representative experiments.

(C) Primary AML cell samples (n=3) were plated in a methylcellulose colony forming assay with increasing concentrations of flubendazole. Colonies were counted 7 days after plating and normalized to cultures treated with buffer alone. Data represent the mean±SD from 3 independent experiments performed in duplicate.

FIG. 2: Flubendazole delays tumor growth and reduces tumor weight in leukemia and myeloma mouse xenografts

Sub-lethally irradiated SCID mice were injected subcutaneously with OCI-AML2 cells (n=20; 10 per group). After implantation, mice were treated with 50 mg/kg flubendazole (A), 20 mg/kg flubendazole (B), or vehicle control by intraperitoneal injection daily. Tumor volume was measured over time. After 16 days (20 mg/kg dose) or 18 days (50 mg/kg dose), mice were sacrificed and tumors were excised, measured and weighted.

(C) Sub-lethally irradiated SCID mice were injected subcutaneously with OPM2 cells (n=20; 10 per group). One week after implantation, when tumors were palpable, mice were treated with 50 mg/kg flubendazole or vehicle control by intraperitoneal injection twice daily. Tumor volume and body weight was measured over time. After 17 days, mice were sacrificed and tumors were excised, measured and weighted.

Data are presented as means±SEM. Differences in tumor volume and tumor weight were analyzed by an unpaired t-test: ***p<0.0001; **p<0.001; *p<0.05.

FIG. 3: Flubendazole inhibits tubulin structure, polymerization and function

(A) Flubendazole (100 μM) and vinblastine (10 μM) were incubated with bovine tubulin (1.5 μM) and the conformational changes were monitored spectrophotometrically by measuring the decrease in the number of reactive cysteine residues at an absorbance of 412 nm as described in the materials and methods. A representative figure is shown.

(B) Flubendazole (100 μM), colchicine (6.0 μM) and taxol (6.0 μM) were incubated with bovine tubulin (1.8 mg/ml) and the effects on polymerization were monitored spectrophotometrically by measuring turbidity at 340 nm as described in the materials and methods. A representative figure is shown.

(C) Tubulin (5.0 μM) was incubated for 30 min with 100 μM vinblastine, 100 μM flubendazole, or buffer control. After incubation, colchicine (10 μM) was added and incubated for 60 minutes. Fluorescence of the tubulin-colchicine complex was measured with excitation and emission wavelengths of 360 nm and 430 nm, respectively. Reduced fluorescence indicates binding at the colchicine site. *p<0.01 (ANOVA, bonferoni post hoc). A representative figure is shown.

(D) PPC-1 cells were treated with 1.0 μM flubendazole (i) or control (ii) for 24 h and stained with DAPI and an anti α-tubulin AlexaFluor 488 nm antibody. Images were captured using an Olympus Fluorview confocal microscope at room temperature. Representative confocal micrographs at 40× are shown.

(E) HeLa cells were grown to confluence and a wound created on the cell monolayer using a 200 μL pipette. Cells were treated with increasing concentrations of flubendazole and imaged every 2 h for 8 h. Wound healing was measured as described in the materials and methods. Representative data is shown and are presented as % wound recovery. *p<0.05 (ANOVA, bonferroni post hoc).

FIG. 4: Flubendazole induces cell cycle arrest and mitotic catastrophe

OCI-AML2 cells (A) or PPC-1 cells (B) were incubated with 1.0 μM flubendazole or buffer control for 24 h. Cells were then stained with PI and the DNA content was measured by flow cytometry. A representative figure is shown.

(C) PPC-1 cells were treated as above and stained with anti α-tubulin antibody and DAPI, as described in the materials and methods. Cells were imaged by confocal microscopy and the number of multi-nucleated cells was enumerated. Data represent the mean±SD percent of multinucleated cells from a representative experiment *p<0.0001 (unpaired t test). Insert: a representative multi-nucleated cell.

FIG. 5: Flubendazole synergizes with vinblastine and vincristine

The effects of increasing concentrations of flubendazole in combination with vinblastine (A) or colchicine (B) on the viability of OCI-AML2 cells. Cell viability was measured by the MTS assay after 72 h incubation. Data were analyzed with Calcusyn software as described in ‘Materials and Methods’. Combination index (CI) versus Fractional effect (Fa) plot showing the effect of the combination of flubendazole and vinblastine or colchicine. CI<1 indicates synergism. One of two representative isobologram experiments performed in triplicate is shown.

Sub-lethally irradiated SCID mice were injected subcutaneously with OCI-AML2 cells (n=40; 10 per group). After implantation, mice were treated with (C) 15 mg/kg flubendazole, 0.3 mg/kg vinblastine, a combination of flubendazole and vinblastine, or vehicle control; or (D) 20 mg/kg flubendazole, 0.25 mg/kg vincristine, a combination of flubendazole and vincristine, or vehicle control. After 16 (C) or 18 (D) days, mice were sacrificed and tumors were excised, measured and weighted. Data represent the mean±SD tumor weight. A representative experiment is shown. *p<0.05, *p<0.01, **p<0.001 (Unpaired t-test).

FIG. 6: Flubendazole does not alter glucose uptake

(A) OCI-AML2 cells were incubated with increasing concentrations of flubendazole for 16 h and the uptake of 3H-deoxy-D-glucose was measured as in the materials and methods. Data represent the mean±SD glucose uptake. A representative experiment is shown.

(B) OCI-AML2 cells were grown in media supplemented with increasing concentrations of glucose in the presence or absence of 1.0 μM flubendazole for 72 h. After incubation, cell growth and viability was measured by the MTS assay. Data represent the mean±SD percent viable cells. A representative experiment is shown.

FIG. 7: Drug treatments do not affect mouse body weight

(A) Mice injected with flubendazole (20 mg/kg), vincristine (0.25 mg/kg) or the combination of flubendazole and vincristine does not affect mouse body weight after 18 days of treatment. (B) Mice injected with flubendazole (15 mg/kg), vinblastine (0.3 mg/kg) or the combination of flubendazole and vinblastine does not affect mouse body weight after 16 days of treatment.

### DETAILED DESCRIPTION OF THE INVENTION

Drugs with previously unrecognized anti-cancer activity could be rapidly repurposed for this new indication given their prior toxicity testing. To identify such compounds, chemical screens were conducted which identified the antihelmintic flubendazole. Flubendazole induced cell death in leukemia and myeloma cell lines and primary patient samples at nanomolar concentrations. Moreover, it delayed tumor growth in leukemia and myeloma xenografts without evidence of toxicity. Mechanistically, flubendazole inhibited tubulin polymerization by binding tubulin at a site distinct from vinblastine. In addition, cells resistant to vinblastine due to over-expression of p-glycoprotein (Pgp) remained fully sensitive to flubendazole, indicating that flubendazole can overcome some forms of vinblastine resistance. Given the different mechanisms of action, the combination of flubendazole and vinblastine was evaluated in vitro and in vivo. Flubendazole synergized with vinblastine to reduce the viability of OCI-AML2 cells. In addition, combinations of flubendazole with vinblastine or vincristine in a leukemia xenograft model delayed tumor growth more than either drug alone. Therefore, flubendazole is a novel microtubule inhibitor that displays therapeutic activity in leukemia and myeloma. Given its prior safety record when evaluated for the treatment of gastrointestinal parasites, flubendazole can be repurposed for the treatment of patients with hematologic malignancies.

### I. DEFINITIONS

The term “flubendazole” as used herein refers to a compound having the formula:

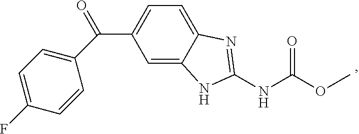

or a pharmaceutically acceptable solvate or prodrug thereof.

Flubendazole has been sold under the brand names Flubenol®, Biovermin®, Flubenol KH®, Flumoxal® and Flutelmium®. Methods of making flubendazole are known in the art and described for example in U.S. Pat. No. 3,657,267, herein incorporated by reference.

The term “vinca alkaloids” as used herein refers to a group of antimitotic chemotherapeutic drugs that can be isolated from the periwinkle plant (Vinca rosea) and includes without limitation, vinblastine, vincristine, vindesine and vinorelbine as well as mixtures thereof.

The term “vinblastine”, also known as “vincaleukoblastine”, as used herein refers a compound having the formula:

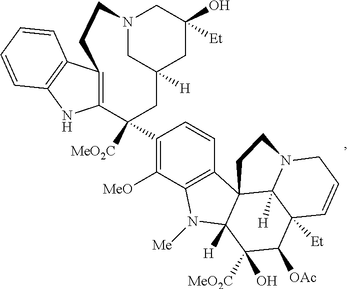

or a pharmaceutically acceptable salt, solvate or prodrug thereof.

Vinblastine can be isolated or synthesized using methods known in the art, for example using methods described in U.S. Pat. No. 5,034,320. Pharmaceutically acceptable salts include for example vinblastine sulfate. Vinblastine is available for example under the brand name Velbe®.

The term “vincristine”, also known as “leurocristine”, as used herein refers to a compound having the formula:

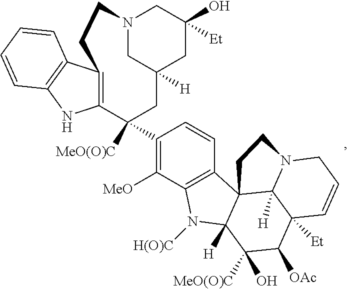

or a pharmaceutically acceptable salt, solvate or prodrug thereof.

Vincristine can be isolated or synthesized using methods known in the art, for example as described in U.S. Pat. No. 4,767,855, and references cited therein. Pharmaceutically acceptable salts include for example vincristine sulfate. Vincristine is available for example under the brand name Oncovin®.

The term “vindesine” as used herein refers to a compound having the formula:

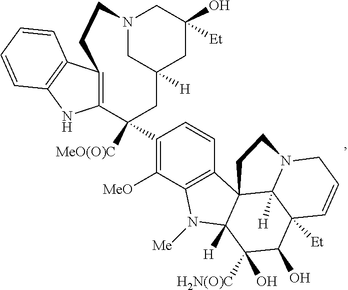

or a pharmaceutically acceptable salt, solvate or prodrug thereof.

Vindesine can be isolated or synthesized using methods known in the art including for example as described in U.S. Pat. No. 4,210,584 and references cited therein. Pharmaceutically acceptable salts include for example vindesine sulfate. Vindesine is sold for example under the brand name Eldisine®.

The term “vinorelbine” as used herein refers to a compound having the formula:

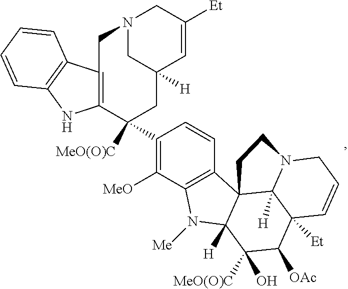

or a pharmaceutically acceptable salt, solvate or prodrug thereof.

Vinolrelbine can be isolated or synthesized using methods known in the art including for example as described in Fahy, J. et al. Biorg. Med. Chem. Lett. 2002, 12:505-507. Pharmaceutically salts include for example vinolrebine tartrate. Vinolrebine is sold for example under the brand name Navelbine®.

The term “cell death” as used herein includes all forms of cell death including for example necrosis and apoptosis.

The term “pharmaceutically acceptable salt” means an acid or basic addition salt, which is suitable for or compatible with the treatment of patients.

The term “pharmaceutically acceptable acid addition salt” as used herein means any non-toxic organic or inorganic salt of any basic compound. Basic compounds that form an acid addition salt include, for example, compound comprising an amine group. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts can be formed, and such salts may exist in either a hydrated, solvated or substantially anhydrous form. In general, acid addition salts are more soluble in water and various hydrophilic organic solvents, and generally demonstrate higher melting points in comparison to their free base forms. The selection of the appropriate salt will be known to one skilled in the art.

The term “pharmaceutically acceptable basic addition salt” as used herein means any non-toxic organic or inorganic base addition salt of any acidic compound. Acidic compounds that form a basic addition salt include, for example, compounds comprising a carboxylic acid group. Illustrative inorganic bases which form suitable salts include lithium, sodium, potassium, calcium, magnesium or barium hydroxide. Illustrative organic bases which form suitable salts include aliphatic, alicyclic or aromatic organic amines such as methylamine, trimethylamine and picoline, alkylammonias or ammonia. The selection of the appropriate salt will be known to a person skilled in the art.

The formation of a desired compound salt is achieved using standard techniques. For example, the neutral compound is treated with an acid or base in a suitable solvent and the formed salt is isolated by filtration, extraction or any other suitable method.

The term “solvate” as used herein means a compound or its pharmaceutically acceptable salt, wherein molecules of a suitable solvent are incorporated in the crystal lattice. A suitable solvent is physiologically tolerable at the dosage administered. Examples of suitable solvents are ethanol, water and the like. When water is the solvent, the molecule is referred to as a “hydrate”. The formation of solvates will vary depending on the compound and the solvate. In general, solvates are formed by dissolving the compound in the appropriate solvent and isolating the solvate by cooling or using an antisolvent. The solvate is typically dried or azeotroped under ambient conditions.

In general, prodrugs will be functional derivatives of parent compounds which are readily convertible in vivo into the parent compound from which it is notionally derived. Prodrugs include, for example, conventional esters formed with an available, carboxylic acid, hydroxy and/or amino group. For example, available OH and/or NH₂ groups in a compounds is acylated using an activated acid in the presence of a base, and optionally, in inert solvent (e.g. an acid chloride in pyridine). Some common esters which have been utilized as prodrugs are phenyl esters, aliphatic (C₈-C₂₄) esters, acyloxymethyl esters, carbamates and amino acid esters. In certain instances, prodrugs are those in which the carboxylic acid, hydroxy and/or amino groups in a compound is masked as a group which can be converted to carboxylic acid, hydroxy and/or amino groups in vivo. Conventional procedures for the selection and preparation of suitable prodrugs are described, for example, in “Design of Prodrugs” ed. H. Bundgaard, Elsevier, 1985.

As used herein, the phrase “effective amount” or “therapeutically effective amount” means an amount effective, at dosages and for periods of time necessary to achieve the desired result. For example in the context or treating a hematological malignancy, an effective amount is an amount that for example induces remission, reduces tumor burden, and/or prevents tumor spread or growth compared to the response obtained without administration of the compound(s). Effective amounts may vary according to factors such as the disease state, age, sex, weight of the subject. The amount of a given compound that will correspond to such an amount will vary depending upon various factors, such as the given drug or compound, the pharmaceutical formulation, the route of administration, the type of disease or disorder, the identity of the subject or host being treated, and the like, but can nevertheless be routinely determined by one skilled in the art.

The term “treating” or “treatment” as used herein and as is well understood in the art, means an approach for obtaining beneficial or desired results, including clinical results. Beneficial or desired clinical results include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease, and remission (whether partial or total), whether detectable or undetectable. “Treating” and “Treatment” can also mean prolonging survival as compared to expected survival if not receiving treatment. “Treating” and “treatment” as used herein also include prophylactic treatment. For example, a subject with early stage multiple myeloma is treated to prevent progression or alternatively a subject in remission is treated to prevent recurrence. Treatment methods comprise administering to a subject a therapeutically effective amount of one or more compounds or compositions described in the present disclosure and optionally consists of a single administration, or alternatively comprises a series of applications. For example, the compounds or compositions described herein are administered at least once a week, from about one time per week to about once daily to about four times daily for a given treatment. The length of the treatment period depends on a variety of factors, such as the severity of the disease, the age of the patient, the concentration, the activity of the compound(s), and/or a combination thereof. It will also be appreciated that the effective dosage of the compound used for the treatment or prophylaxis may increase or decrease over the course of a particular treatment or prophylaxis regime. Changes in dosage may result and become apparent by standard diagnostic assays known in the art. In some instances, chronic administration may be required.

The dosage administered will vary depending on the use and known factors such as the pharmacodynamic characteristics of the particular compound, and its mode and route of administration, age, health, and weight of the individual recipient, nature and extent of symptoms, kind of concurrent treatment, frequency of treatment, and the effect desired. Dosage regime may be adjusted to provide the optimum therapeutic response.

The term “subject” as used herein includes all members of the animal kingdom including mammals, and suitably refers to humans.

The term “hematological malignancy” or “hematological cancer” as used herein refers to cancers that affect blood and bone marrow.

The term “hematological cancer cell” as used herein refers a cancerous cell of the blood and bone marrow lineages, including primary cells. Hematological cancer cells include for example leukemia cells such as leukemia cells represented by CEM, TEX, THP1, HL-60, RSV411, K562, Jurkat, U937, OCI-M2, OCI-AML2 and NB4 leukemia cell lines and cells phenotypically similar thereto, lymphoma cells such as lymphoma cells represented MDAY-D2 and cell phenotypically similar thereto, and multiple myeloma cells such as multiple myeloma cells represented by OPM2, KMS11, LP1, UTMC2, KSM18, KSM12, H929, JJN3 and OCIMy5 myeloma cell lines and cells phenotypically similar thereto. Hematological cancer cells also include chronic myelogenous leukemia cells, including cells representing the blast crises phases such as K562 and cells phenotypically similar thereto; AML cells such as represented by HL-60, K562, OCI-M2, and NB4 and cells phenotypically similar thereto, ALL cells such as represented by RSV411 and Jurkat and cells phenotypically similar thereto, and lymphoma cells such as represented by MDAY-D2 and cells phenotypically similar thereto.

The term “leukemia” as used herein means any disease involving the progressive proliferation of abnormal leukocytes found in hemopoietic tissues, other organs and usually in the blood in increased numbers. For example, leukemia includes acute myeloid leukemia (AML), acute lymphocytic leukemia (ALL) and chronic myelogenous leukemia (CML).

The term “lymphoma” as used herein means any disease involving the progressive proliferation of abnormal lymphoid cells. For example, lymphoma includes mantle cell lymphoma, Non-Hodgkin's lymphoma, and Hodgkin's lymphoma. Non-Hodgkin's lymphoma would include indolent and aggressive Non-Hodgkin's lymphoma. Aggressive Non-Hodgkin's lymphoma would include intermediate and high grade lymphoma. Indolent Non-Hodgkin's lymphoma would include low grade lymphomas.

The term “myeloma” and/or “multiple myeloma” as used herein means any tumor or cancer composed of cells derived from the hemopoietic tissues of the bone marrow. Multiple myeloma is also knows as MM and/or plasma cell myeloma.

The term “phenotypically similar” refers to a cell type that exhibits morphological, physiological and/or biochemical characteristics similar to another cell type. For example, a cell that is phenotypically similar to an AML cell can include a cell that comprises Auer rods. As another example, U937 cells which are derived from a patient with lymphoma, show morphological similarity to monocytoid AML cells. As a further example the leukemia cell line NB4 differentiates similar to promyelocytic cells (PML) with all trans retinoic acid (ATRA) and thereby represents a “phenotypically similar” model of PML cells.

As used herein, “contemporaneous administration” and “administered contemporaneously” means that two substances are administered to a subject such that they are both biologically active in the subject at the same time. The exact details of the administration will depend on the pharmacokinetics of the two substances in the presence of each other, and can include administering one substance within 24 hours of administration of the other, if the pharmacokinetics are suitable. Designs of suitable dosing regimens are routine for one skilled in the art. In particular embodiments, two substances will be administered substantially simultaneously, i.e. within minutes of each other, or in a single composition that comprises both substances.

The term “combination therapy” as used herein means two or more substances are administered to a subject over a period of time, contemporaneously or sequentially e.g. the substances can for example be administered at the same time or at different times within the period of time, at similar or different intervals. The compounds may or may not be biologically active in the subject at the same time. As an example, a first substance is administered weekly, and a second substance administered every other week for a number of weeks. The exact details of the administration will depend on the pharmacokinetics of the two substances. Designs of suitable dosing regimens are routine for one skilled in the art.

The term “pharmaceutically acceptable” means compatible with the treatment of animals, in particular, humans.

The term “synergistic” as used herein means the enhanced or magnified effect of a combination on at least one property compared to the additive individual effects of each component of the combination. For example, compounds that induce cell death by the same mechanism, e.g. binding tubulin at the same site would not be expected to have more than additive effect. Synergism can be assessed and quantified for example by analyzing the Data by the Calcusyn median effect model where the combination index (CI) indicates synergism (CI<0.9), additively (CI=0.9-1.1) or antagonism (CI>1.1). CIs of <0.3, 0.3-0.7, 0.7-0.85, 0.85-0.90, 0.90-1.10 or >1.10 indicate strong synergism, synergism, moderate synergism, slight synergism, additive effect or antagonism, respectively. The CI is the statistical measure of synergy.

The term “drug resistant” as used herein refers to a cancer or cancer cell, particularly a hematological malignancy or hematological cancer cell that resists the effects of chemotherapy, e.g. that is able to survive in the presence of a concentration of drug compared to a suitable comparator cell. A drug resistant cancer or cancer cell for example can require increased concentration of drug for the drug to be effective. A cell or cancer can become resistant to a drug. For example, a cancer cell can mutate, amplify a gene that renders the drug ineffective, pump the drug out of the cell for example via P-glycoprotein (Pgp), inactivate the drug, prevent transport of the drug into the cell or prevent the cell from dying after exposure to the drug.

In the following passages, different aspects are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Moreover, all percentages are based on weight percentages unless otherwise specified. Further, as used in this specification and the appended claims, the singular forms “a”, “an” and “the” include plural references unless the content clearly dictates otherwise. Thus for example, a composition containing “a compound” includes a mixture of two or more compounds. It should also be noted that the term “or” is generally employed in its sense including “and/or” unless the content clearly dictates otherwise.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. Finally, terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

Finally, all references cited herein are incorporated by reference.

### II. METHODS/USES

Therapeutic regimens for treating hematological malignancies are herein described.

Flubendazole is demonstrated herein to be cytotoxic to leukemic and myeloma cells, and to have anti-tumor effects in vivo without causing weight loss, behavioural changes or gross organ toxicity in mice. Accordingly, an aspect of the disclosure includes a method of treating a hematological malignancy in a subject in need thereof comprising administering to the subject an effective amount of flubendazole and/or a pharmaceutically acceptable solvate and/or prodrug thereof.

Combination therapies are also disclosed. Accordingly, another aspect of the disclosure includes a method of treating a hematological malignancy in a subject in need thereof comprising administering to the subject an effective amount of flubendazole and/or a pharmaceutically acceptable solvate and/or prodrug thereof, and an effective amount of a vinca alkaloid and/or a pharmaceutically acceptable salt, solvate and/or prodrug thereof.

Also included in an aspect of the disclosure is a use of flubendazole and/or a pharmaceutically acceptable solvate and/or prodrug thereof for treating a hematological malignancy.

Yet another aspect includes a use of flubendazole and/or a pharmaceutically acceptable salt, solvate and/or prodrug thereof, in combination with a vinca alkaloid and/or a pharmaceutically acceptable salt, solvate and/or prodrug thereof for treating a hematological malignancy.

A further aspect includes a use of flubendazole and/or a pharmaceutically acceptable solvate and/or prodrug thereof for the manufacture of a medicament for treating a hematological malignancy. Another aspect includes a use of flubendazole and/or a pharmaceutically acceptable solvate and/or prodrug thereof, in combination with a vinca alkaloid and/or a pharmaceutically acceptable salt, solvate and/or prodrug thereof for the manufacture of a medicament for treating a hematological malignancy.

Another aspect of the disclosure relates to a method of inducing cell death of a hematological cancer cell comprising contacting the cancer cell with an effective amount of flubendazole and/or a pharmaceutically acceptable solvate and/or prodrug thereof alone or in combination with a vinca alkaloid and/or a pharmaceutically acceptable salt, solvate and/or prodrug thereof.

Also included is a method of inhibiting tubulin polymerization in a cell comprising contacting a cell with an effective amount of flubendazole and/or a solvate and/or prodrug thereof, alone or in combination with a vinca alkaloid and/or a salt, solvate and/or prodrug thereof. Inhibiting tubulin polymerization means reducing polymerized tubulin in a cell or sample by at least 50%, at least 60%, at least 70%, at least 80%, at least 90% or at least 95% compared to a suitable control, such as an untreated cell or sample as determined using assays known in the art.

In an embodiment, the flubendazole is administered before the vinca alkaloid. In another embodiment, the flubendazole is administered after the vinca alkaloid. In yet another embodiment, the flubendazole is administered simultaneously with the vinca alkaloid. In yet another embodiment, the flubendazole is administered contemporaneously with the vinca alkaloid. In embodiments, the compounds are administered in a single dose or in multiple applications, at similar or different intervals, for example flubendazole is administered daily and a vinca alkaloid is administered once or twice weekly for a particular number or weeks.

In an embodiment, the vinca alkaloid is selected from vinblastine, vincristine, vindesine, and vinolrebine and pharmaceutically acceptable salts, solvates and prodrugs thereof and mixtures thereof.

In an embodiment, the pharmaceutically acceptable salt of the vinca alkaloid is a sulfate salt or a tartrate salt.

In an embodiment, the hematological malignancy or hematological cancer cell is drug resistant to a vinca alkaloid. In a further embodiment, the hematological malignancy or hematological cancer cell overexpresses P-glycoprotein (Pgp). In an embodiment, the method comprises first identifying subjects having a hematological malignancy resistant to a vinca alkaloid and then administering to the subject an effective amount of flubendazole and/or a pharmaceutically acceptable solvate and/or prodrug thereof, and optionally an effective amount of a vinca alkaloid and/or a pharmaceutically acceptable salt, solvate and/or prodrug thereof. For example, a subject with a hematological malignancy who has failed treatment with a vinca alkaloid such as vinblastine or vincristine is identified as a subject having a hematological malignancy resistant to a vinca alkaloid. As an alternative example, a sample of the hematological malignancy and/or a cancer cell is tested for drug resistance, for example vinca alkaloid resistance.

Vinca alkaloids such as vincristine are currently used in the treatment of leukemia and myeloma however neurotoxicity is a dose limiting toxicity of vincristine. As flubendazole strongly synergized with vinca alkaloids in cell and animal studies it is expected that the same anti-tumor effect can be obtained with lower concentrations of the vinca alkaloids when combined with flubendazole and/or the combination would provide better anti-tumor efficacy without increased toxicity.

Accordingly, an aspect includes a method of reducing toxicity associated with the administration of a vinca alkaloid comprising administering an effective amount of flubendazole and/or a pharmaceutically acceptable solvate and/or prodrug thereof in combination with a vinca alkaloid and/or a pharmaceutically acceptable salt, solvate and/or prodrug thereof, wherein the quantity of the vinca alkaloid and/or a pharmaceutically acceptable salt, solvate and/or prodrug thereof administered is reduced compared to a standard treatment protocol.

Standard treatment protocols for vinca alkaloids are well known in the art. For example, vinblastine can be administered between about 3.7 mg/m² bsa and about 18.5 mg/m² bsa and vincristine can be administered at about 1.4 mg/m² bsa or about up to 2 mg/m² bsa.

The reduction can for example be about 5%, 10%, 15%, 20%, 25%, 30% or more reduction.

A further aspect includes a method of increasing anti-tumor efficacy of a vinca alkaloid comprising administering an effective amount of flubendazole and/or a pharmaceutically acceptable solvate and/or prodrug thereof in combination with a vinca alkaloid and/or a pharmaceutically acceptable salt, solvate and/or prodrug thereof.

In another embodiment, the hematological malignancy is a leukemia. In another embodiment, the leukemia is selected from acute myeloid leukemia (AML), acute lymphocytic leukemia (ALL) and chronic myeloma leukemia (CML). In an embodiment, the hematological cancer cell is leukemic cell, an AML cell, an ALL cell or a CML cell.

In a further embodiment, the hematological malignancy is a myeloma. In another embodiment, the hematological cancer cell is a myeloma cell.

In yet a further embodiment, the hematological malignancy is a lymphoma. In an embodiment, the hematological cancer cell is a lymphoma cell.

In yet a further aspect, the disclosure includes methods and uses wherein the compound and/or compounds administered are selected from flubendazole and/or a pharmaceutically acceptable solvate and/or prodrug thereof alone or in combination with a vinca alkaloid and/or a pharmaceutically acceptable salt, solvate and/or prodrug thereof, and are administered at a dosage and/or comprised dosage form described herein, for example an oral dosage form or an intravenous dosage form.

### III. COMPOSITIONS, COMBINATION PRODUCTS AND KITS

Compositions for treating hematological malignancies are herein provided.

An aspect of the disclosure includes a composition comprising an effective amount of flubendazole, and/or a pharmaceutically acceptable prodrug and/or or solvate thereof, and a suitable carrier or vehicle, for treating hematological malignancies.

Another aspect of the disclosure includes a composition comprising a flubendazole, and/or a pharmaceutically acceptable prodrug and/or solvate thereof; a vinca alkaloid and/or a pharmaceutically acceptable salt, prodrug and/or solvate thereof; and, optionally, a suitable carrier or vehicle.

In an embodiment, the vinca alkaloid is selected from vincristine, vinblastine, vindesine and vinorelbine, and pharmaceutically acceptable salts, solvates and prodrugs thereof and combinations thereof.

In an embodiment, the pharmaceutically acceptable salt of the vinca alkaloid is a sulfate salt or a tartrate salt.

Another aspect of the disclosure includes a composition comprising an effective amount of flubendazole, and/or a pharmaceutically acceptable prodrug and/or solvate thereof; a vinca alkaloid and/or a pharmaceutically acceptable salt, prodrug and/or solvate thereof; and a suitable carrier or vehicle for treating a hematological malignancy.

The compounds are suitably formulated into pharmaceutical compositions for administration to human subjects in a biologically compatible form suitable for administration in vivo.

The disclosure in an aspect, also describes a pharmaceutical composition comprising an effective amount of flubendazole, and/or a pharmaceutically acceptable prodrug and/or solvate thereof; a vinca alkaloid and/or a pharmaceutically acceptable salt, prodrug and/or solvate thereof; and, optionally, a pharmaceutically acceptable carrier, for treatment of a leukemia, lymphoma and/or multiple myeloma.

The compositions described herein can be prepared by per se known methods for the preparation of pharmaceutically acceptable compositions that can be administered to subjects, such that an effective quantity of the active substance is combined in a mixture with a pharmaceutically acceptable vehicle.

Suitable vehicles are described, for example, in Remington's Pharmaceutical Sciences (2003-20^{th} Edition). On this basis, the compositions include, albeit not exclusively, solutions of the substances in association with one or more pharmaceutically acceptable vehicles or diluents, and contained in buffered solutions with a suitable pH and iso-osmotic with the physiological fluids.

Pharmaceutical compositions include, without limitation, lyophilized powders or aqueous or non-aqueous sterile injectable solutions or suspensions, which optionally further contain antioxidants, buffers, bacteriostats and solutes that render the compositions substantially compatible with the tissues or the blood of an intended recipient. Other components that are optionally present in such compositions include, for example, water, surfactants (such as Tween™), alcohols, polyols, glycerin and vegetable oils. Extemporaneous injection solutions and suspensions may be prepared from sterile powders, granules, tablets, or concentrated solutions or suspensions. The composition may be supplied, for example but not by way of limitation, as a lyophilized powder which is reconstituted with sterile water or saline prior to administration to the subject.

Suitable pharmaceutically acceptable carriers include essentially chemically inert and nontoxic compositions that do not interfere with the effectiveness of the biological activity of the pharmaceutical composition. Examples of suitable pharmaceutical carriers include, but are not limited to, water, saline solutions, glycerol solutions, ethanol, N-(1(2,3-dioleyloxy)propyl)N,N,N-trimethylammonium chloride (DOTMA), diolesyl-phosphotidyl-ethanolamine (DOPE), and liposomes. Such compositions should contain a therapeutically effective amount of the compound(s), together with a suitable amount of carrier so as to provide the form for direct administration to the subject.

In an embodiment, the compositions described herein are administered for example, by parenteral, intravenous, subcutaneous, intramuscular, intracranial, intraorbital, ophthalmic, intraventricular, intracapsular, intraspinal, intracisternal, intraperitoneal, intranasal, aerosol or oral administration.

Compositions for nasal administration may conveniently be formulated as aerosols, drops, gels and powders. Aerosol formulations typically comprise a solution or fine suspension of the active substance in a physiologically acceptable aqueous or non-aqueous solvent and are usually presented in single or multidose quantities in sterile form in a sealed container, which can take the form of a cartridge or refill for use with an atomizing device. Alternatively, the sealed container may be a unitary dispensing device such as a single dose nasal inhaler or an aerosol dispenser fitted with a metering valve which is intended for disposal after use. Where the dosage form comprises an aerosol dispenser, it will contain a propellant which can be a compressed gas such as compressed air or an organic propellant such as fluorochlorohydrocarbon. The aerosol dosage forms can also take the form of a pump-atomizer.

Wherein the route of administration is oral, the dosage form may be for example, incorporated with excipient and used in the form of enteric coated tablets, caplets, gelcaps, capsules, ingestible tablets, buccal tablets, troches, elixirs, suspensions, syrups, wafers, and the like. The oral dosage form may be solid or liquid.

Accordingly, a further aspect of the disclosure is a composition formulated for as an oral dosage form selected from enteric coated tablets, caplets, gelcaps, and capsules, each unit dosage form comprising about 10 to less than about 5000 mg, suitably about 10 to about 3500 mg, about 10 to about 1500 mg, about 10 to about 1200 mg, about 10 to about 1000 mg, about 10 to about 800 mg, about 10 to about 500 mg, about 10 to about 300 mg, about 50 to about 3500 mg, about 50 to about 1500 mg, about 50 to about 1200 mg, about 50 to about 1000 mg, about 50 to about 800 mg, about 50 to about 500 mg, about 50 to about 300 mg, about 30 to about 300 mg, or about 35 to about 50 mg, of one or more compounds selected from flubendazole and/or a pharmaceutically acceptable solvate and/or prodrug thereof and/or a vinca alkaloid and/or a pharmaceutically acceptable salt, solvate and/or prodrug thereof and a pharmaceutically acceptable carrier.

In an embodiment, the disclosure describes a pharmaceutical composition wherein the dosage form is a solid dosage form. A solid dosage form refers to individually coated tablets, capsules, granules or other non-liquid dosage forms suitable for oral administration. It is to be understood that the solid dosage form includes, but is not limited to, modified release, for example immediate release and timed-release, formulations. Examples of modified-release formulations include, for example, sustained-release (SR), extended-release (ER, XR, or XL), time-release or timed-release, controlled-release (CR), or continuous-release (CR or Contin), employed, for example, in the form of a coated tablet, an osmotic delivery device, a coated capsule, a microencapsulated microsphere, an agglomerated particle, e.g., as of molecular sieving type particles, or, a fine hollow permeable fiber bundle, or chopped hollow permeable fibers, agglomerated or held in a fibrous packet. Timed-release compositions can be formulated, e.g. liposomes or those wherein the active compound is protected with differentially degradable coatings, such as by microencapsulation, multiple coatings, etc. It is also possible to freeze-dry the compounds of the disclosure and use the lyophilizates obtained, for example, for the preparation of products for injection.

Accordingly, a further aspect of the disclosure is a pharmaceutical composition in solid dosage form comprising about 10 to less than about 5000 mg, suitably about 10 to about 3500 mg, about 10 to about 1500 mg, about 10 to about 1200 mg, about 10 to about 1000 mg, about 10 to about 800 mg, about 10 to about 500 mg, about 10 to about 300 mg, about 50 to about 3500 mg, about 50 to about 1500 mg, about 50 to about 1200 mg, about 50 to about 1000 mg, about 50 to about 800 mg, about 50 to about 500 mg, about 50 to about 300 mg, about 30 to about 300 mg, or about 35 to about 50 mg, of one or more compounds selected from flubendazole and/or a pharmaceutically acceptable solvate and/or prodrug thereof and/or a vinca alkaloid and/or a pharmaceutically acceptable salt, solvate and/or prodrug thereof and a pharmaceutically acceptable carrier.

Compositions suitable for buccal or sublingual administration include tablets, lozenges, and pastilles, wherein the active ingredient is formulated with a carrier such as sugar, acacia, tragacanth, and/or gelatin and/or glycerin.

In another embodiment, the disclosure describes a pharmaceutical composition wherein the dosage form is a liquid oral dosage form. A person skilled in the art would know how to prepare suitable formulations. Conventional procedures and ingredients for the selection and preparation of suitable formulations are described, for example, in Remington's Pharmaceutical Sciences (2003-20th edition) and in The United States Pharmacopeia The National Formulary (USP 24 NF19) published in 1999.

Accordingly, a further aspect of the disclosure is a pharmaceutical composition in oral liquid dosage form comprising about 10 to less than about 5000 mg, suitably about 10 to about 3500 mg, about 10 to about 1500 mg, about 10 to about 1200 mg, about 10 to about 1000 mg, about 10 to about 800 mg, about 10 to about 500 mg, about 10 to about 300 mg, about 50 to about 3500 mg, about 50 to about 1500 mg, about 50 to about 1200 mg, about 50 to about 1000 mg, about 50 to about 800 mg, about 50 to about 500 mg, about 50 to about 300 mg, about 30 to about 300 mg, or about 35 to about 50 mg, of one or more compounds selected from flubendazole and/or a pharmaceutically acceptable solvate and/or prodrug thereof and/or a vinca alkaloid and/or a pharmaceutically acceptable salt, solvate and/or prodrug thereof and a pharmaceutically acceptable carrier.

In another embodiment, the disclosure describes a pharmaceutical composition wherein the dosage form is an injectable dosage form. An injectable dosage form is to be understood to refer to liquid dosage forms suitable for, but not limited to, intravenous, subcutaneous, intramuscular, or intraperitoneal administration. Solutions of compounds described herein can be prepared in water suitably mixed with a surfactant such as hydroxypropylcellulose. Dispersions can also be prepared in glycerol, liquid polyethylene glycols, DMSO and mixtures thereof with or without alcohol, and in oils. Under ordinary conditions of storage and use, these preparations contain a preservative to prevent the growth of microorganisms. A person skilled in the art would know how to prepare suitable formulations. Conventional procedures and ingredients for the selection and preparation of suitable formulations are described, for example, in Remington's Pharmaceutical Sciences (2003-20th edition) and in The United States Pharmacopeia: The National Formulary (USP 24 NF19) published in 1999.

Accordingly, a further aspect of the disclosure is a pharmaceutical composition in injectable dosage form comprising about 10 to less than about 5000 mg, suitably about 10 to about 3500 mg, about 10 to about 1500 mg, about 10 to about 1200 mg, about 10 to about 1000 mg, about 10 to about 800 mg, about 10 to about 500 mg, about 10 to about 300 mg, about 50 to about 3500 mg, about 50 to about 1500 mg, about 50 to about 1200 mg, about 50 to about 1000 mg, about 50 to about 800 mg, about 50 to about 500 mg, about 50 to about 300 mg, about 30 to about 300 mg, or about 35 to about 50 mg, of one or more compounds selected from flubendazole and/or a pharmaceutically acceptable solvate and/or prodrug thereof and/or a vinca alkaloid and/or a pharmaceutically acceptable salt, solvate and/or prodrug thereof and a pharmaceutically acceptable carrier.

The pharmaceutical forms suitable for injectable use include sterile aqueous solutions or dispersion and sterile powders for the extemporaneous preparation of sterile injectable solutions or dispersions. In all cases the form must be sterile and must be fluid to the extent that easy syringability exists.

In an embodiment, the dosage form comprises about 10 mg to about 5000 mg of one or more compounds selected from flubendazole and/or a pharmaceutically acceptable solvate and/or prodrug thereof. In another embodiment, the dosage form comprises about 10 mg to about 1500 mg of one or more compounds selected from flubendazole and/or a pharmaceutically acceptable solvate and/or prodrug thereof. In yet another embodiment, the dosage form comprises about 30 mg to about 300 mg of one or more compounds selected from flubendazole and/or a pharmaceutically acceptable solvate and/or prodrug thereof. In other embodiments, the dosage form comprises about 10 to about 3500 mg, about 10 to about 1200 mg, about 10 to about 1000 mg, about 10 to about 800 mg, about 10 to about 500 mg, about 10 to about 300 mg, about 50 to about 3500 mg, about 50 to about 1500 mg, about 50 to about 1200 mg, about 50 to about 1000 mg, about 50 to about 800 mg, about 50 to about 500 mg, about 50 to about 300 mg, about 30 to about 300 mg, or about 35 to about 50 mg, of one or more of one or more compounds selected from flubendazole and/or a pharmaceutically acceptable solvate and/or prodrug thereof.

In one embodiment the dosage, for example the daily dosage, comprises about 20 mg to about 5000 mg of one or more compounds described herein. In another embodiment, the dosage comprises about 100 mg to about 1500 mg of one or more compounds described herein. In yet another embodiment, the dosage comprises about 400 to about 1200 mg of one or more compounds described herein. In other embodiments, the dosage form comprises about 10 to about 3500 mg, about, about 10 to about 1200 mg, about 10 to about 1000 mg, about 10 to about 800 mg, about 10 to about 500 mg, about 10 to about 300 mg, about 50 to about 3500 mg, about 50 to about 1500 mg, about 50 to about 1200 mg, about 50 to about 1000 mg, about 50 to about 800 mg, about 50 to about 500 mg, about 50 to about 300 mg, about 30 to about 300 mg, or about 35 to about 50 mg of one or more compounds described herein.

The dosage form may alternatively comprise about 1 to about 200 mg of one or more compounds described herein/kg body weight, about 2 to about 100 mg of one or more compounds described herein/kg body weight, about 20 to about 100 mg of one or more compounds described herein/kg body weight, about 5 to about 50 mg of one or more compounds described herein/kg body weight about 5 to about 25 mg of one or more compounds described herein/kg body weight, or about 5 to about 15 mg of one or more compounds described herein/kg body weight of a subject in need of such treatment formulated into a solid oral dosage form, a liquid oral dosage form, or an injectable dosage form. In an embodiment, the dosage comprises about 5 to about 15 mg of one or more compounds described herein/kg body weight of a subject in need of such treatment formulated into a solid oral dosage form, a liquid dosage oral form, or an injectable dosage form.

In an embodiment, the dosage form comprises an effective amount or a therapeutically effective amount. In one embodiment the dosage form comprises about 10 to about 5000 mg of one or more compounds described herein. In another embodiment, the dosage form comprises about 10 to about 1500 mg of the compound(s). In another embodiment, the dosage form comprises about 30 to about 300 mg of the compound(s).

The flubendazole and/or a pharmaceutically acceptable solvate and/or prodrug thereof and the vinca alkaloid and/or a pharmaceutically acceptable salt, solvate and/or prodrug thereof are optionally in the same dosage form or in different dosage forms. For example flubendazole is optionally in an oral dosage form and the vinca alkaloid is an injectable dosage form.

Also included in another aspect of the disclosure, is a kit comprising flubendazole and/or a pharmaceutically acceptable solvate and/or prodrug thereof and a vinca alkaloid and/or a pharmaceutically acceptable salt, solvate and/or prodrug thereof for treating a hematological malignancy.

Another embodiment includes a kit comprising flubendazole and/or a pharmaceutically acceptable solvate and/or prodrug thereof and instructions for use in combination with a vinca alkaloid and/or a pharmaceutically acceptable salt, solvate and/or prodrug thereof for treating a hematological malignancy.

Another embodiment includes a kit comprising a vinca alkaloid and/or a pharmaceutically acceptable salt, solvate and/or prodrug thereof and instructions for use in combination with flubendazole and/or a pharmaceutically acceptable solvate and/or prodrug thereof for treating a hematological malignancy.

The following non-limiting examples are illustrative of the present disclosure:

### EXAMPLES

### Example 1

To date, the identification of drugs with unanticipated anti-cancer effects has been largely serendipitous. Disclosed herein is a systematic approach to identify compounds with unanticipated anti-cancer activity by testing libraries of drugs in chemical screens. From these screens, flubendazole, a member of the benzimidazole family of antihelmintic drugs, was identified to have anti-leukemia and anti-myeloma activity. Flubendazole has been extensively evaluated in humans and animals for the treatment of intestinal parasites as well as for the treatment of systemic worm infections. In these studies, patients have received up to 50 mg/kg orally daily for 24 months without serious adverse effects.¹²⁻¹⁴ Healthy volunteers have also received single oral doses up to 2000 mg without toxicity.¹⁵ In sheep receiving a single flubendazole dose of 5 mg/kg intravenously, no toxicity was observed and the area under the curve (AUC) was 6.53 μg·h/mL (22 μM).¹⁶

While selected members of the benzimidazole family have recently been reported to induce cell death in solid tumor cell lines,¹⁷⁻¹⁹ the anti-tumor properties of flubendazole have not been previously reported. Moreover, the mechanism by which benzimidazoles exert their effects as antihelmintics and by which they induce cell death in malignant cells is not fully understood and several cellular responses have been described. For example, benzimidazoles have been shown to inhibit amino peptidase activity and glutamate catabolism, reduce glucose uptake, increase intracellular calcium levels, and inhibit microtubule formation.²⁰⁻²²

Flubendazole displays anti-leukemia and anti-myeloma activity in vitro and in vivo at pharmacologically achievable concentrations. Mechanistically, flubendazole inhibits tubulin structure and function by interacting with a site on tubulin distinct from vinca alkaloid tubulin inhibitors. In keeping with this different mechanism of tubulin inhibition, flubendazole synergized with vinca alkaloids, vinblastine and vincristine to induce cell death and delay tumor growth in vivo. Therefore, given its prior safety and toxicity testing, flubendazole could be rapidly repurposed as a novel agent for the treatment of leukemia and myeloma for use in combination with vinca alkaloids.

### Results

A Screen of Drugs for Compounds with Novel Anti-Cancer Activity Identifies Flubendazole

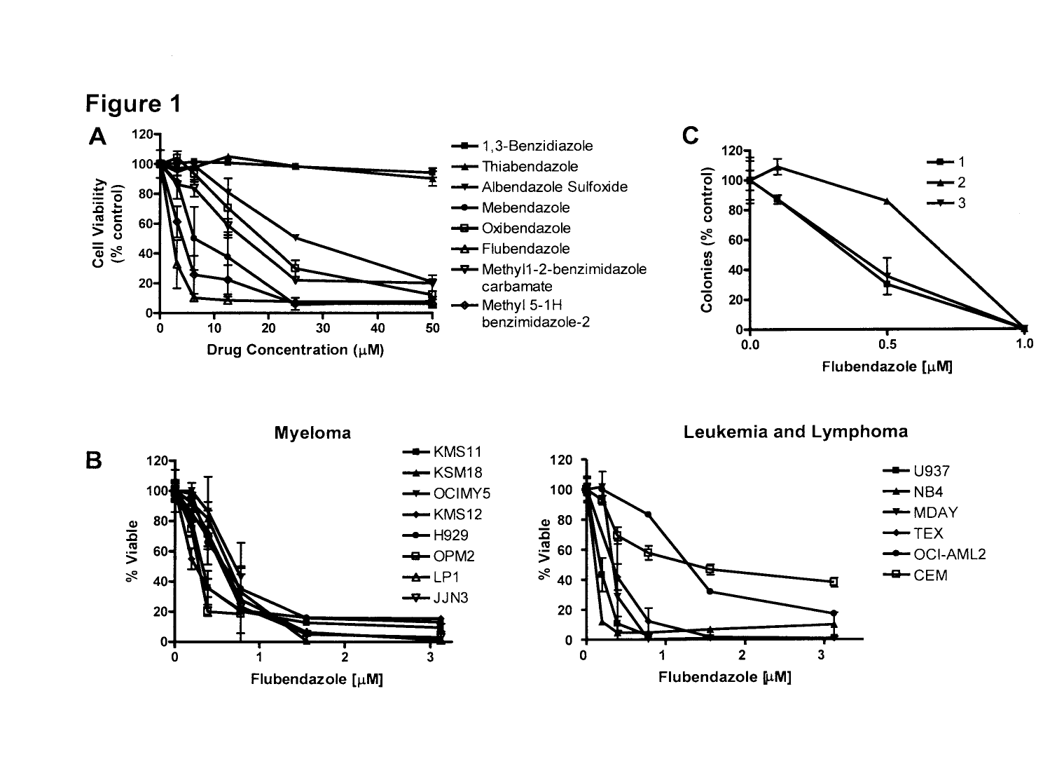

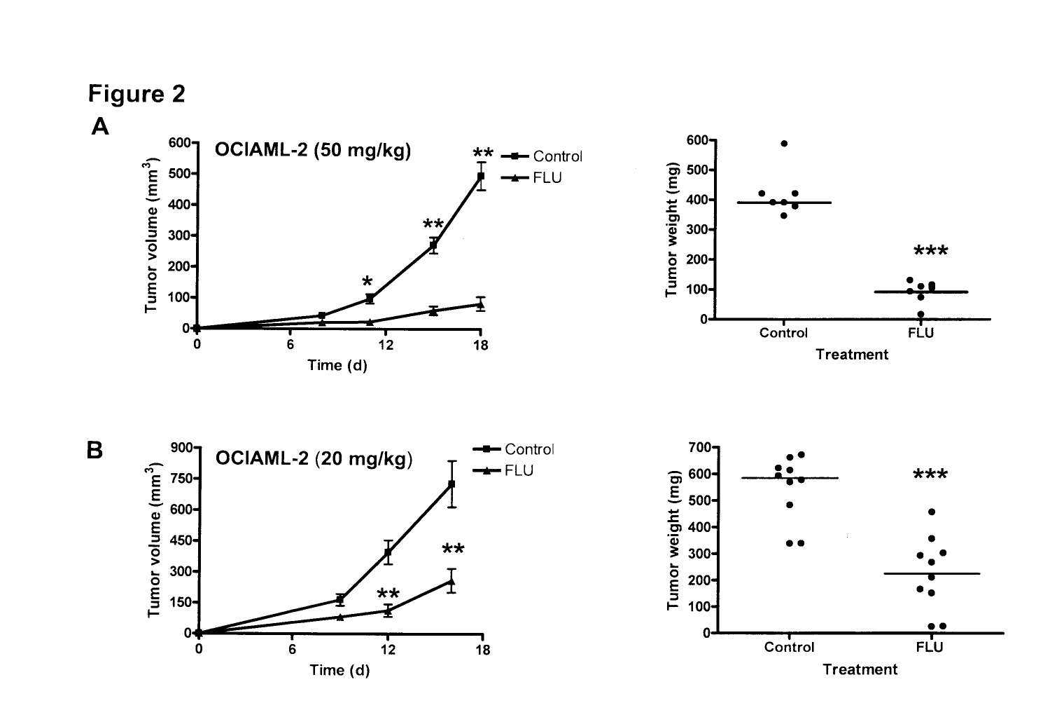

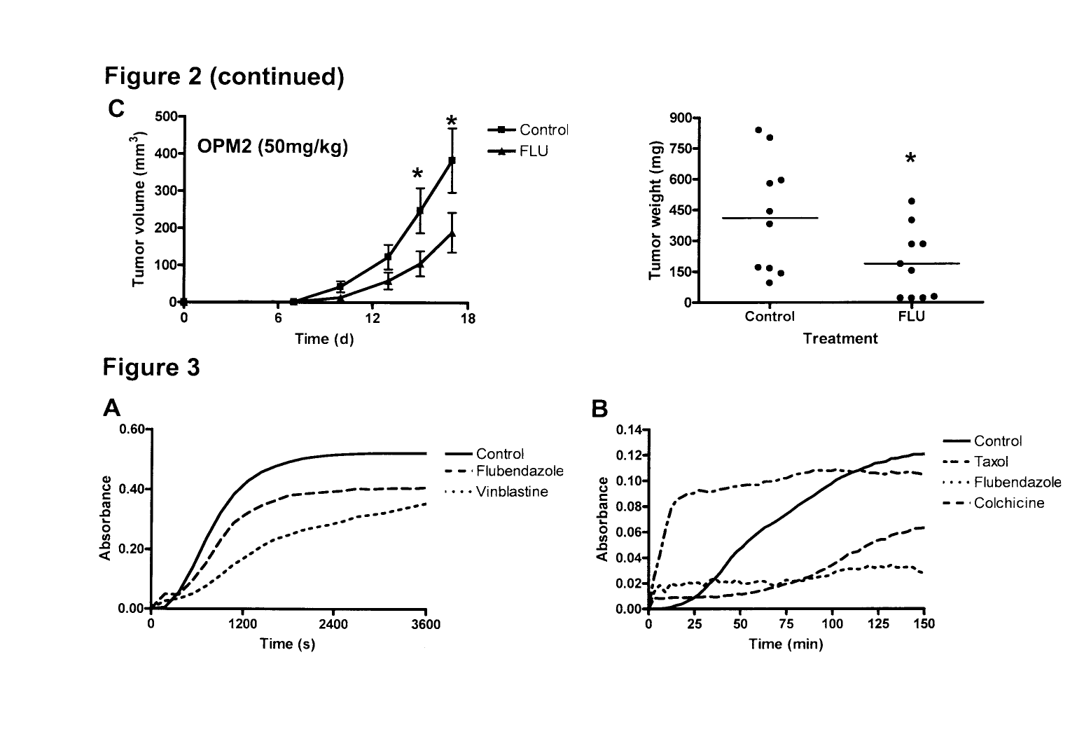

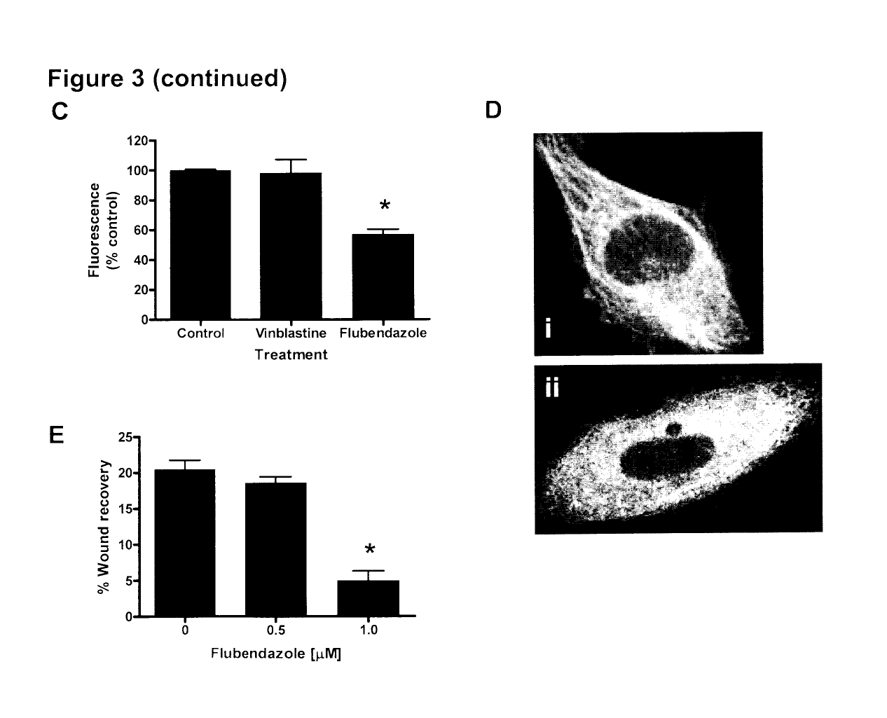

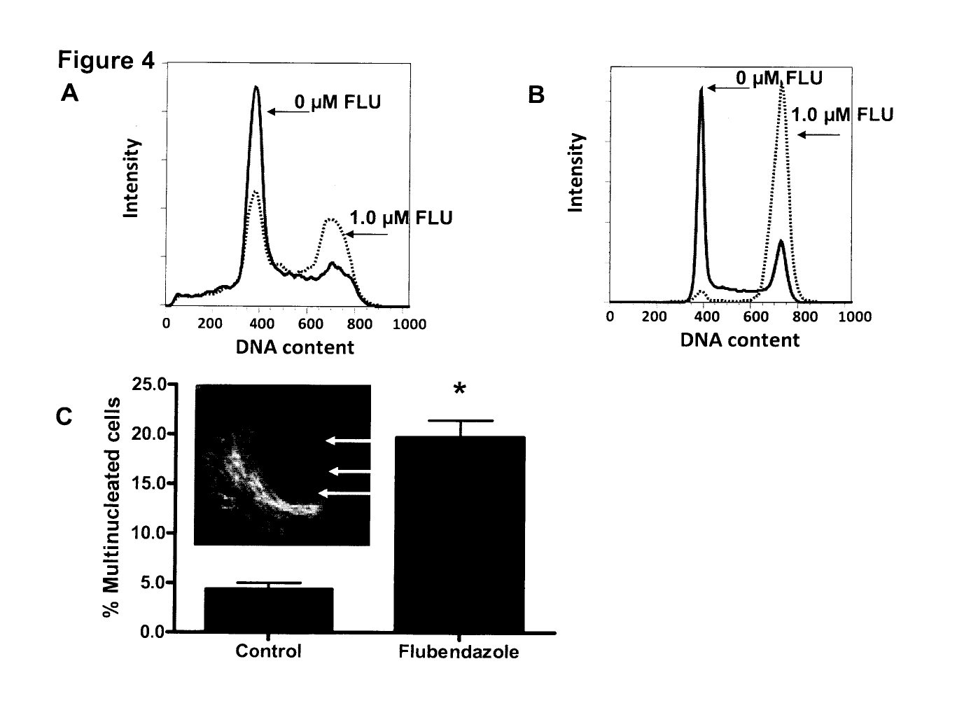

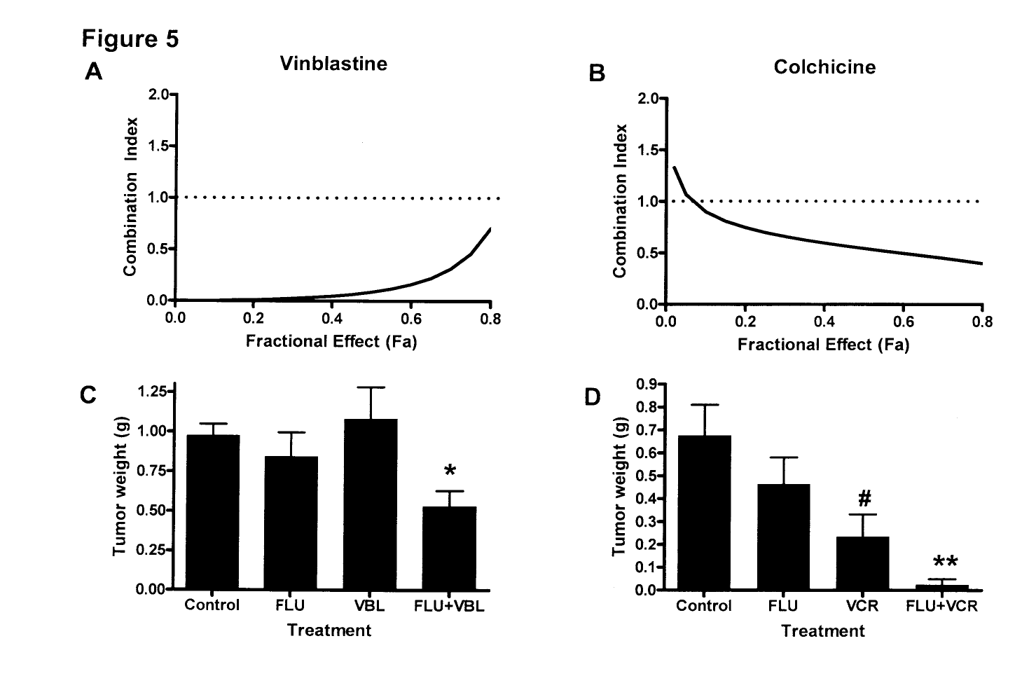

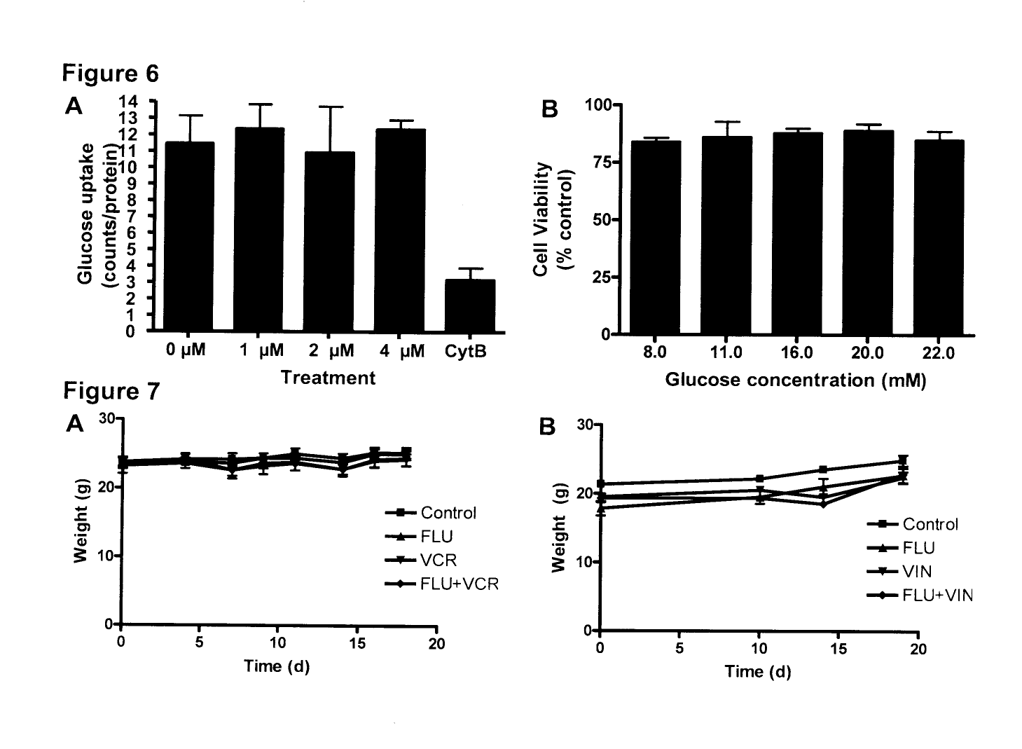

To identify drugs with unanticipated anti-cancer activity, a library of 110 drugs focused on anti-microbials and metabolic regulators with a wide therapeutic index and well characterized pharmacokinetics was compiled. This library was then screened to identify compounds that were cytotoxic to leukemia cell lines³⁴ and identified several cytotoxic agents including mebendazole. Mebendazole is a member of the benzimidazole family of antihelmintics, so the cytotoxicity of this drug class was investigated. OCI-AML2 leukemia cells were treated with increasing concentrations of 8 benzimidazoles. Seventy two hours after incubation, cell growth and viability was measured by the MTS assay. The most potent benzimidazole in this panel was flubendazole (FIG. 1A).

### Flubendazole is Cytotoxic to Leukemia and Myeloma Cell Lines

Having identified flubendazole as a potential anti-cancer agent, its effects were evaluated in a panel of malignant cell lines. Leukemia and myeloma cell lines were treated with increasing concentrations of flubendazole. Seventy two hours after incubation, cell growth and viability was measured by the MTS assay. Flubendazole reduced cell viability with an LD₅₀≦1.0 μM in 8/8 myeloma and 4/6 leukemia cell lines, including MDAY-D2 cells with an LD₅₀ of 3.0 nM (FIG. 1B). Of note, the remaining 2 cells lines (OCI-AML-2 and CEM) had LD₅₀ values of 1.07±0.6 and 1.9±0.9 μM respectively. Concentrations of 1.0 μM appear pharmacologically achievable, based on prior studies that demonstrated an AUC of 22 μM in the absence of toxicity.¹⁶ Cell death was confirmed by PI staining and the trypan blue exclusion assay. Flubendazole also reduced the clonogenic growth of primary AML samples (FIG. 1C). Thus, flubendazole displays activity against leukemia and myeloma cells at nanomolar concentrations.

### Flubendazole Delays Tumor in Leukemia and Myeloma Xenografts

As flubendazole was cytotoxic to malignant cells in vitro, its anti-tumor effects were evaluated in leukemia and myeloma xenografts. OCI-AML2 leukemia cells were injected subcutaneously into sub-lethally irradiated SCID mice. Mice were then treated with flubendazole (20 or 50 mg/kg) daily or vehicle control intraperitoneally. Compared to mice treated with vehicle control, flubendazole significantly delayed tumor growth and reduced tumor weights (p<0.0001; FIGS. 2A-B). Likewise, sub-lethally irradiated SCID mice were injected subcutaneously with OPM2 myeloma cells. Mice were then treated daily with 50 mg/kg of flubendazole or buffer control for 17 days intraperitoneally. Flubendazole significantly delayed tumor growth and reduced tumor weights compared to mice treated with vehicle control (p<0.05; FIG. 2C).

In both leukemia and myeloma models, there was no evidence of weight loss, behavioral changes or gross organ toxicity with flubendazole treatment. Thus, flubendazole displays novel pre-clinical activity against leukemia and myeloma.

Flubendazole does not Alter Glucose Uptake

In studies with the parasite Trichuris globulosa, the antihelmintic effects of the benzimidazoles thiabendazole and fenbendazole were related to inhibition of glucose uptake with resultant alterations in glucose metabolism.²¹ Therefore, we tested the effects of flubendazole on glucose uptake in malignant cells. OCI-AML2 cells were treated with increasing concentrations of flubendazole for 16 hours and uptake of 3H-deoxy-D-glucose was measured (FIG. 6A). In contrast to the observations in the parasite, flubendazole at concentrations up to 4.0 μM did not alter glucose uptake. Likewise, culturing cells with varying concentrations of glucose did not alter flubendazole-induced death (FIG. 6B). Thus, flubendazole-induced cell death does not appear related to inhibition of glucose uptake.

### Flubendazole Alters Microtubule Structure and Function

To better understand the mechanism by which flubendazole induced death of malignant cells changes in gene expression following 4 hours of flubendazole treatment were examined. By gene ontology and pathway analysis, 196 genes were identified to be deregulated >4 fold with flubendazole treatment (ArrayExpress accession: E-MEXP-2352; Table 2). Of these, 179 genes were annotated and 58/179 fell within 8 functional annotations associated with chromosomal segregation and cytoskeleton regulation; genes involved in chromosomal segregation were the most affected by flubendazole treatment (Table 3). Moreover, by Connectivity Map analysis, changes in gene expression were found to be similar to gene signatures induced by the known tubulin inhibitors nocodazole and colchicine.

The effects of flubendazole on tubulin structure and polymerization were evaluated. To determine whether flubendazole alters tubulin structure, changes in the number of reactive cysteine residues on tubulin following incubation with flubendazole were measured. Treatment with flubendazole reduced the number of reactive cysteines by 5.2±2.6 compared to buffer control (p<0.05). In comparison, vinblastine decreased the number of reactive cysteines by 8.6±2.5 (p<0.01). Thus, these results suggest that flubendazole interacts with tubulin to alter its structure (FIG. 3A).

Drugs that alter microtubules can either promote or inhibit tubulin polymerization.³⁵ Therefore, the effects of flubendazole on tubulin polymerization were investigated. Flubendazole was incubated with purified bovine tubulin and tubulin polymerization was recorded over time. As controls, bovine tubulin was incubated with colchicine which is known to inhibit tubulin polymerization³⁶ and taxol which is known to promote tubulin polymerization.³⁷ In this assay, flubendazole inhibited tubulin polymerization (FIG. 3B).

As flubendazole altered the structure and polymerization of tubulin, its binding site to vinblastine, a member of the vinca alkaloid family of microtubule inhibitors that is currently used for the treatment of leukemia and myeloma was compared.^{38,39} Purified bovine tubulin was incubated with flubendazole or vinblastine followed by the addition of colchicine. The interaction of colchicine with tubulin was then assayed by measuring the fluorescence of colchicine bound to tubulin.⁴⁰ The addition of flubendazole decreased colchicine fluorescence, but the addition of vinblastine had no effect (FIG. 3C), indicating that flubendazole, but not vinblastine, blocks colchicine from binding tubulin. Thus flubendazole interacts with tubulin through a mechanism distinct from vinblastine.

Since flubendazole altered tubulin structure and function in cell-free assays, the effects of flubendazole on microtubule formation in intact cells were evaluated. PPC-1 prostate cancer cells were treated with flubendazole (1.0 μM) or vehicle control for 24 hours, and then stained with anti-tubulin and DAPI. Microtubule architecture was visualized by confocal microscopy (FIG. 3D). PPC-1 cells treated with vehicle control exhibited an organized network of elongated microtubules (FIG. 3Di). In contrast, cells treated with flubendazole were rounded with contracted and disorganized microtubules (FIG. 3Dii). Thus, flubendazole disrupts the microtubule architecture in intact cells.

Microtubules mediate cell migration⁴¹, the effects of flubendazole on cell migration with a wound healing assay were investigated. HeLa cells were seeded in 4 well chambers and after adhering overnight, the cell monolayer was scratched to create a wound. Cells were treated with flubendazole or buffer control and migration of cells to heal the wound was measured overtime. Treatment with flubendazole impaired cell migration and delayed wound healing (FIG. 3E). Of note, at the concentrations and times tested in this these assays, the flubendazole-treated cells were more than 89% viable as measured by MTS assay.

### Flubendazole Arrests Cells in Cell Cycle and Induces Mitotic Catastrophe

Inhibition of tubulin polymerization can inhibit cell cycle progression and induce mitotic catastrophe⁴², so the effects of flubendazole on the cell cycle by flow cytometry and on chromosomal segregation by enumerating the number of multi-nucleated cells in OCI-AML2 and PPC-1 cells were assessed. Flubendazole arrested cells in the G2 phase of the cell cycle (FIGS. 4A-B) and increased the number of multi-nucleated cells (FIG. 4C). Thus, flubendazole produces cell cycle arrest and mitotic catastrophe, consistent with its effects as a microtubule inhibitor.

### Inhibition of Microtubules is Functionally Important for Flubendazole's Cytotoxicity

Next, whether microtubule inhibition was functionally important for flubendazole's cytotoxicity was determined. KB-4.0-HTI cells have a single nucleotide change in α-tubulin that renders it resistant to tubulin inhibitors.²³ Therefore, we treated KB-4.0-HTI and KB-3-1 wild type controls with increasing concentrations of flubendazole and colchicine. Consistent with flubendazole's effects on tubulin, KB-4.0-HTI cells were more resistant to flubendazole with an IC₅₀ 7-fold higher than the non-mutated KB-3-1 wild type cells (Table 1). Similarly, KB-4.0-HTI cells were also resistant to colchicine with an IC₅₀ 2.5-fold higher than the KB-3-1 control cells (Table 1), consistent with previous observations with this cell line.²³ Flubendazole was also tested in A549.EpoB40 cells, which are more sensitive to microtubule inhibitors due to a point mutation in β-tubulin at residue 292.²⁴ A549.EpoB40 cells were more sensitive to flubendazole with an IC₅₀ 5-fold lower than the non-mutated A549 control cells (Table 1). Similarly, A549.EpoB40 cells were sensitive to colchicine with an IC₅₀ 1.5-fold lower than the A549 control cells (Table 1), consistent with previous observations with this cell line.²⁴ As further evidence that flubendazole's cytotoxicity was related to microtubule inhibition, the benzimidazoles thiabendazole and 1,3-benzidiazole that did not induce cell death in OCI-AML2 cells (FIG. 1A) also did not inhibit microtubule formation in our cell-free polymerization assays. Thus, taken together, flubendazole induces cell death through a mechanism that appears related to its inhibition of microtubule polymerization.

Over-Expression of P-Glycoprotein does not Alter Flubendazole's Cytotoxicity.

Over-expression of P-glycoprotein (Pgp, MDR1) renders cells resistant to vinca alkaloid microtubule inhibitors.⁴³ Therefore, the effects of Pgp over-expression on flubendazole's cytotoxicity were tested. CEM-VBL cells over-express Pgp,⁴⁴ which was confirmed with a rhodamine dye exclusion assay. CEM wild type and CEM-VBL cells were treated with increasing concentrations of flubendazole or vinblastine and cell viability measured by the MTS assay (Table 1). CEM-VBL cells remained fully sensitive to flubendazole compared to the wild type controls. In contrast, CEM-VBL cells over-expressing Pgp were resistant to vinblastine at concentrations greater than 5.0 μM (Table 1). Therefore, over-expression of Pgp does not abrogate the cytotoxicity of flubendazole and this drug is capable of over-coming some forms of resistance to vinca alkaloids.

Flubendazole does not Induce Neuropathy

Neuropathy is a dose limiting toxicity of vinca alkaloid microtubule inhibitors such as vincristine. Therefore, the effects of flubendazole on neurologic function in mice were tested. Mice (n=10/group) were treated with 50, 100, and 200 mg/kg of flubendazole or vehicle control intraperitoneally daily for 14 days, and sensory function was assessed with the tail-flick assay. No changes in tail-flick latency were observed at doses up to 200 mg/kg compared to controls, a dose 10-fold higher than the dose required for an anti-tumor effect (mean±SD tail-flick latency: control 3.04±0.52 seconds vs. 200 mg/kg flubendazole 2.91±0.50 seconds, p=0.58 by Student's t-test). However, doses of 200 mg/kg resulted in 5% decrease in body weight compared to vehicle controls (p=0.03, Student's t-test).

Flubendazole Synergizes with Vinblastine and Enhances Vinblastine and Vincristine Activity In Vivo

Flubendazole interacts with tubulin through a mechanism distinct from vinblastine. Therefore, the cytotoxicity of flubendazole and vinblastine in combination was evaluated. OCI-AML2 cells were treated with increasing concentrations of flubendazole and vinblastine and 72 h after incubation cell growth and viability was measured by the MTS assay. Combinations were assessed based on CI values where CI values <1, equal to 1 or >1 are considered synergistic, additive or antagonistic, respectively.^{33,45} The combination of flubendazole and vinblastine synergistically induced cell death with CI values of 0.09, 0.017, 0.003 and 0.001 at the EC 50, 25, 10 and 5, respectively (FIG. 5A). In contrast, cell death produced by the combination of flubendazole and colchicine was closer to additive with CI values of 0.54, 0.70, 0.897 and 1.07 at EC 50, 25, 10 and 5, respectively (FIG. 5B).

Given the synergy of flubendazole and vinblastine in cell culture, the combination of flubendazole and vinblastine and vincristine in vivo was evaluated. OCI-AML2 cells were injected subcutaneously into SCID mice and treated intraperitoneally with flubendazole (15 mg/kg), vinblastine (0.3 mg/kg) or vincristine (0.25 mg/kg or 0.35 mg/kg), or the combination of the two agents. The combination of flubendazole and vinblastine decreased tumor weight greater than either agent alone (p<0.01) (FIG. 5C). Similarly, the combination of flubendazole and vincristine decreased tumor weight greater than either agent alone (p<0.001) (FIG. 5D). Moreover, there were no observed behavioral changes, weight loss, or gross organ toxicity from either combination treatment (FIG. 7). Thus, flubendazole synergizes with vinblastine and vincristine and could be used in combination with these vinca alkaloids to achieve a greater anti-tumor effect.

### Discussion

Through screens of libraries of drugs flubendazole was identified as having previously unrecognized anti-leukemia and anti-myeloma activity. At pharmacologically achievable concentrations, flubendazole induced cell death in malignant cells and delayed tumor growth in vivo. Mechanistically, flubendazole altered microtubule structure and inhibited tubulin polymerization by interacting with a site on tubulin similar to colchicine and distinct from vinblastine.

As part of its development as an antihelmintic, flubendazole has been studied extensively in animals and humans, where it has displayed favorable toxicology profiles. For example, in rats, mice and guinea-pigs the LD₅₀ is >5000 mg/kg and >400 mg/kg after oral and intraperitoneal administration, respectively.¹⁵ No toxicity was noted in rats that received up to 150 mg/kg/day for 3 months, while chickens receiving up to 180 mg/kg flubendazole daily for 7 days developed anemia and reduction of red cells in the spleen.¹⁵ In humans, doses of 40-50 mg/kg/day for 10 days have been administered for the treatment of neurocysticercosis and no toxicity from the drug was reported.¹⁴ Likewise, patients received up to 50 mg/kg/day of flubendazole for up to 24 months for the treatment of alveolar echinococcosis without adverse effect.¹³

The pharmacokinetics of flubendazole are also well characterized. For example, in sheep the estimated half life for flubendazole after oral administration is 6.5 hours and the main metabolic pathways are carbamate hydrolysis and ketone reduction.¹⁵ After intravenous administration, an AUC of 22 μM is achieved over 36 hours after a single intravenous dose of 5 mg/kg. However, only 18% of flubendazole is absorbed, so after a single oral dose of 5 mg/kg the AUC over 36 hours was 1.17 μg·h/mL (4.0 μM).¹⁶ Similar pharmacokinetics are observed in healthy human volunteers and patients receiving flubendazole for the treatment of parasitic infection. For example, in healthy volunteers who received 1000 mg flubendazole as a single oral dose, 77% of unchanged drug was detected in the feces and less than 0.1% in the urine three days after administration.¹⁵ Thus, taken together, flubendazole has poor oral bioavailability. However, since large doses can be safely administered and since oral administration of flubendazole has clinical efficacy in the treatment of systemic worm infections, oral flubendazole would be expected to be useful for anti-cancer activity. Alternatively, an intravenous formulation would be useful.

In support of the evaluation of flubendazole for the treatment of patients with refractory hematologic malignancies, a Phase I clinical trial of the related benzimidazole, albendazole was recently conducted in patients with advanced solid tumors.⁴⁶ In 2 of 7 patients, albendazole reduced levels of the tumor markers AFP and CEA. However, albendazole caused severe neutropenia in 3 of these patients and the development of albendazole as an anti-tumor agent has not been pursued to date.

In the present study, flubendazole inhibited tubulin polymerization and function which were functionally important for its cytotoxicity. Microtubules are cytoskeleton components that are required for cell division, cellular transport and in the maintenance of cellular integrity.³⁵ Microtubules are comprised of α- and β-tubulin heterodimers that assemble into linear protofilaments and polymerize into hollow, cylindrical structures.³⁸ The gain or loss of tubulin heterodimers leads to elongation or shortening of the microtubules.⁴⁷ Drugs that alter the polymerization of microtubule are well validated therapeutic agents for the treatment of malignancies. For example, vinca alkaloids are used in the treatment of leukemia and myeloma and inhibit microtubule polymerization by binding to β-tubulin near the GTP-binding site.³⁸ In contrast, colchicine inhibits polymerization by binding the interface of the α/β-tubulin heterodimer and taxol promotes tubulin polymerization by binding in the lumen of the polymer.³⁸ In our study, we demonstrated that flubendazole interacts with tubulin at a site similar to that of colchicine and distinct from vinca-alkaloids. A similar interaction with tubulin has been reported for the benzimidazole mebendazole.⁴⁸ However, the benzimidazole benomyl has been reported to inhibit tubulin polymerization by interacting at a site distinct from both the colchicine site and the vinca domain.¹⁹ Thus, the mechanism by which benzimidazoles inhibit tubulin formation appears to vary among family members.

Vinca alkaloids are currently used in the treatment of leukemia and myeloma, and neurotoxicity is a dose limiting toxicity of vincristine. In contrast, flubendazole did not produce acute neurotoxicity as evaluated in the tail-flick assay. However, additional neurotoxicology testing with more prolonged dosing could fully evaluate the sensory and motor effects of this drug. It is noted that prior animal toxicology studies and human clinical trials have not reported neurotoxicity after flubendazole administration.

As flubendazole and vinca alkaloids inhibited tubulin through distinct mechanisms, the combination of these drugs was evaluated. Flubendazole synergized with the vinca alkaloids vinblastine and vincristine in vitro and in vivo. Therefore, these drugs can be used in combination to enhance the efficacy of standard therapy for these diseases or potentially reduce their toxicity.

Finally, as flubendazole inhibits tubulin through a mechanism distinct from vinca alkaloids, flubendazole can be useful in overcoming some forms of resistance to vinca alkaloids. For example, as over-expression of Pgp does not render cells resistant to flubendazole, it could also overcome this specific mechanism of resistance to vinca alkaloids.

In summary, flubendazole is a novel inhibitor of microtubules that acts through a mechanism distinct from vinca alkaloids. Given its prior safety record in humans and animals coupled with its pre-clinical efficacy in hematological malignancies, flubendazole can be repurposed for evaluation in these diseases.

### Materials and Methods

### Reagents

Colchicine, taxol, and the panel of benzimidazole compounds were purchased from Sigma Chemical (St. Louis, Mo.). Vinblastine was purchased from Calbiochem (San Diego, Calif.). Drugs were prepared as stock solutions in dimethyl sulfoxide (DMSO).

### Cell Culture

Leukemia (U937, MDAY, CEM, CEM-VBL) and solid tumor cell lines (PPC-1, HeLa) were cultured in RPMI 1640 medium. The epidermoid carcinoma cell lines KB-3-1 and KB-4.0-HTI²³ were grown in Dulbecco's Modified Eagle Medium. The lung carcinoma cell lines A549 and A549.EpoB40 were grown in RPMI with the latter supplemented with 20% fetal calf serum (FCS), and 40 nM epothilone B.²⁴ Leukemia cell lines OCI-AML2, CEM, and NB4 and all the myeloma cell lines (OPM2, KMS11, JJN3 LP1, H929, L1, KMS12, KSM18 and OCI My5) were maintained in Iscove's Modified Dulbecco's Medium (IMDM). TEX cells were maintained in IMDM, 15% FCS, 1% penicillin-streptomycin, 2 mM L-glutamine, 20 ng/mL SCF, 2 ng/mL IL-3. Unless otherwise noted, all media were supplemented with 10% FCS, 100 units/mL of streptomycin and 100 μg/mL of penicillin (all from Hyclone, Logan, Utah). Cells were incubated in a humidified air atmosphere containing 5% CO₂ at 37° C.

Primary human acute myeloid leukemia (AML) samples were isolated from the peripheral blood of consenting AML patients, who had at least 80% malignant cells among the mononuclear cells in their peripheral blood and cultured at 37° C. in IMDM, 10% fetal calf serum, 100 units/mL of streptomycin and 100 μg/mL of penicillin. The collection and use of human tissue for this study was approved by the local ethics review board (University Health Network, Toronto, ON, Canada).

### Cell Growth and Viability Assays

Cell growth and viability was measured using the 3-(4,5-dimethylthiazol-2-yl)-5-(3-carboxymethoxyphenyl)-2-(4-sulfophenyl)-2H-tetrazolium inner salt (MTS) reduction assay (Promega, Madison, Wis.) according to the manufacture's protocol and as previously described²⁵. Cells were seeded in 96 well plates and treated with drug for 72 h. Optical density (OD) was measured at 490 nm.

Mitotic catastrophe was measured by enumerating the number of multinucleated cells similar to the method previously described²⁶. Briefly, cells (5.0×10⁵) were fixed and stained as described below. Fields containing 100 cells per treatment group were selected at random and visualized at 20×. A total of 700 cells were scored per treatment condition. A positive score for mitotic catastrophe was given when two or more distinct nuclear lobes were visualized within a single cell.

### Analysis of Gene Expression

Changes in gene expression were measured in U937 leukemia cells treated with 1.0 μM flubendazole or buffer control for 4 hours using Ingenuity Pathways Analysis (www.ingenuity.com) and the Database for Annotation, Visualization and Integrated Discovery (DAVID; http://david.abcc.ncifcrf.gov). Gene expression was measured in U937 leukemia cells treated with 1.0 μM flubendazole or buffer control for 4 hours. Briefly, total RNA was harvested from treated and untreated cells and hybridized to Affymetrix HG U133 Plus 2.0 gene expression oligonucleotide arrays (Affymetrix, Santa Clara, Calif., USA). Labeling and hybridization to arrays were performed by The Centre for Applied Genomics (Medical and Related Sciences Centre, Toronto, ON, Canada). Microarray data were analyzed using GeneSpring GX v10.0 (Agilent), and genes deregulated >4-fold after 4 hours flubendazole treatment were identified. Pathways and gene ontology analyses were carried out using Ingenuity Pathways Analysis (www.ingenuity.com) and the Database for Annotation, Visualization and Integrated Discovery (DAVID; http://david.abcc.ncifcrf.gov). Connectivity Map analysis, which compares the changes in gene expression signature following flubendazole treatment with gene expression signatures following treatment with other common pharmacological agents, was also performed (http://www.broadinstitute.org/cmap).

### Leukemia and Myeloma Xenograft Models

Sub-lethally irradiated SCID mice were injected subcutaneously in the left flank with leukemia OCI-AML2 (2.0×10⁶) or myeloma OPM2 (1.0×10⁷) cells. Mice were then randomly assigned to receive flubendazole (in 0.9% NaCl and 0.01% tween-80) or vehicle control (0.9% NaCl and 0.01% tween-80) intraperitoneally. When the combination of flubendazole and vinblastine or vincristine was evaluated, mice were randomly assigned to receive flubendazole (in 0.9% NaCl and 0.01% tween-80), vinblastine (in PBS and 0.01% tween-20) or vincristine (in PBS and 0.01% tween-20), the combination of flubendazole and vinblastine or vincristine, or vehicle control intraperitoneally. Tumor volumes (tumor length×width²×0.5236) were monitored daily using calipers. At the end of the experiment (16-18 days), mice were sacrificed, tumors excised and tumor volume and weight measured. All animal studies were carried out according to the regulations of the Canadian Council on Animal Care and with the approval of the local ethics review board.

### Assessment of Glucose Uptake

The effect of flubendazole on the uptake of 2-deoxy-D-glucose in OCI-AML2 cells was performed using a radioactive glucose uptake assay as described by Wood et al²⁷. OCI-AML2 cells were treated with increasing concentrations of flubendazole or the known glucose uptake inhibitor, cytochalasin B. After treatment, cells were washed twice with a HEPES-buffered saline solution (140 mM NaCl, 5 mM KCl, 2.5 mM MgSO₄, 1 mM CaCl₂, and 20 mM Hepes, pH 7.4). Uptake of 2-deoxy-D-glucose was initiated by the addition of 10 μM 2-deoxy-D-glucose and 1 μCi/mL 2-[³H]deoxy-D-glucose in the HEPES-buffered saline solution at room temperature. After 5 min of incubation, 2-deoxy-D-glucose uptake was terminated by rinsing the cells three times with a 25 mM ice-cold glucose solution, followed by cell disruption with 0.05 N NaOH. Protein concentration was measured by the modified Bradford protein assay and cell-associated radioactivity was determined by scintillation counting. The mean counts per minute are expressed as picomoles/min/mg protein.

### Tubulin Polymerization Assay

Polymerization of bovine tubulin was measured according to Beyer et al²⁸. Briefly, bovine tubulin (1.8 mg/ml; Cytoskeleton; Denver, USA) was added to ice cold polymerization buffer (PEM; 80 mM PIPES, 0.5 mM EGTA, 2 mM MgCl₂, 10% glycerol and 1 mM GTP) and centrifuged at top speed in a microcentrifuge for 5 min at 4° C. Supernatant (100 μL/well) was immediately added to a 96 well plate, which contained colchicine, taxol, flubendazole, or buffer/DMSO control in PEM buffer. Final concentrations of colchicine, taxol and flubendazole were 6.0 μM, 6.0 μM and 100 μM, respectively. Following addition of tubulin, the plate was immediately placed in the spectrophotometer, which was maintained at 37° C., and the absorbance measured every 3 minutes for 2.5 h at 340 nm.

### Measurement of Tubulin Sulfhydryl Groups

The ability of microtubule target drugs to alter tubulin structure was assessed by measuring the number of reactive cysteine residues using the sulfhydryl reagent 5,5′-Dithio-bis(2-nitrobenzoic acid) (DTNB; Sigma) as previously described^{19,29}. Briefly, bovine tubulin (1.5 μM) was incubated with 10 μM vinblastine, 10 μM colchicine, 100 μM flubendazole or a control for 15 min at 4° C. After incubation, DTNB was added (100 μM final concentration) and the absorbance was measured in a 1 cm path length cuvette at 412 nm for 60 min at 37° C. The number of sulfhydryl groups were determined by using a molar extinction coefficient for DTNB of 13 600 (M⁻¹, cm⁻¹).²⁹

### Determining the Site of Binding to Tubulin

The site of flubendazole binding was determined similar to Gupta et al¹⁹. Bovine tubulin (5.0 μM) was incubated with 100 μM flubendazole, 100 μM vinblastine or buffer control for 30 min at 37° C. Colchicine (10 μM) was then added and incubated for an additional 60 min at 37° C. Fluorescence of the colchicine-tubulin complex was subsequently measured at excitation and emission wavelengths of 360 nm and 430 nm, respectively.

### Determining Effect of Flubendazole Microtubules in Cultured Cells

PPC-1 cells (5.0×10³) were seeded on glass cover slips in a 6 well plate overnight at 37° C. and then treated with 1.0 μM flubendazole or vehicle control for 24 h. Cells were then fixed in 3.7% paraformeldehyde for 15 min at 37° C., permeabilized using 0.1% triton X-100 for 4 min at room temperature, and incubated at 4° C. overnight in a humidified chamber with a blocking solution (0.5% BSA). Cells were then incubated with a primary mouse anti α-tubulin antibody (clone DMIA, 1:300, in 0.5% BSA; Sigma) for 1 hour at 37° C., washed 3 times in PBS and subsequently incubated with a goat anti-mouse IgG, AlexaFluor 488 secondary antibody (1:500, Sigma) for 1 hour in the dark at room temperature. Cells were then washed, stained with 4′,6-diamidino-2-phenylindole (DAPI) and mounted on a microscope slide using Dakocytomation fluorescent mounting media (Dakocytamation, Carpinteria, Calif.). Slides were imaged using the Olympus Fluoview FV1000 Laser Scanning Confocal Microscope (Olympus America Inc., Center Valley, Pa.) at room temperature at 40×.

### Cell Migration Assays

HeLa cell migration was measured as previously described³⁰. Briefly, HeLa cells were seeded in 4 well chambers and grown to confluence. A wound was inflicted to the monolayer using a 200 μL pipette tip. Wells were washed with media to remove any detached cells and media containing either 0 μM, 0.5 μM or 1.0 μM flubendazole was then added and incubated at 37° C. for 8 hours. Images were captured using a Zeiss Axiovert 200M microscope every 2 hours. These images were quantified using Image J software, which outlined and enumerated the number of pixels in the wound area. Cell migration of HeLa cells was recorded and calculated as a percent recovery of wound area.

### Cell Cycle Analysis

Cell cycle analysis was performed as described by Mao et al³¹. Briefly, OCI-AML2 or PPC-1 cells were harvested, washed with cold PBS and resuspended in PBS and cold absolute ethanol. Cells were then treated with 100 ng/mL of DNase-free RNase A (Invitrogen, Carlsbad, Calif.) at 37° C. for 30 min, washed with cold PBS, resuspended in PBS and incubated with 50 μg/mL of propidium iodine (PI) for 15 min at room temperature in the dark. DNA content was measured by flow cytometry (FACSCalibur, Becton Dickinson, Florida, USA) and analyzed with FlowJo software version 7.0 (TreeStar, Ashland, Oreg.).

Assessment of Sensory Function with the Tail-Flick Assay

Sensory function was assessed with the tail-flick assay by Chempartners Co. (Shanghai, China) similar to previously described³². Briefly, 50 experimentally-naïve, male, adult C57BU6J mice from Shanghai SLAC Co. Ltd were treated with 50, 100, 200 mg/kg of flubendazole in 0.9% NaCl and 0.01% tween-80 or vehicle control (n=10 per group). Before and after 14 days of treatment, tail flick latency was measured by applying a high-intensity, noxious, radiant heat stimulus 20 mm from the tip of the tail. When a withdrawal tail-flick response occurred, the thermal stimulus was terminated automatically and the response latency was measured electronically.

### Drug Combination Studies

The combination index (CI) was used to evaluate the interaction between flubendazole and colchicine or vinblastine. OCI-AML2 cells were treated with increasing concentrations of flubendazole, vinblastine or colchicine. Seventy-two hours after incubation cell viability was measured by the MTS assay. The Calcusyn median effect model was used to calculate the CI values and evaluate whether the combination of flubendazole with vinblastine or colchicine was synergistic, antagonistic or additive. CI values of <1 indicate synergism, CI=1 indicate additivity and Cl>1 indicate antagonism.³³

### Statistical Analysis

Unless otherwise stated, the results are presented as mean±SD. Data were analyzed using GraphPad Prism 4.0 (GraphPad Software, USA). p<0.05 was accepted as being statistically significant. Drug combination data were analyzed using Calcusyn software (Biosoft, UK).

TABLE 1
Flubendazole effects on mutated cell lines.

| Cell line | Flubendazole (μM) | Colchicine (μM) |
|---|---|---|
| KB-3-1 | 1.9 ± 1.1 | 14.9 ± 4.5 |
| KB-4.0-HTI | 12.5 ± 1.8 | 41.6 ± 5.5 |
| A549 | 4.1 ± 1.3 | 0.09 ± 0.01 |
| A549.EpoB40 | 0.8 ± 0.2 | 0.06 ± 0.01 |
| | Flubendazole (μM) | Vinblastine (μM) |
| CEM | 1.9 ± 0.9 | 0.14 ± 0.01 |
| CEM-VBL | 2.7 ± 1.2 | >5.0 |

Data represent IC₅₀ values, as measured by the MTS assay, calculated from 3 replicates.

TABLE 2
Gene pathway analysis.

| | Fold- | | | |
|---|---|---|---|---|
| | Difference | Deregu- | | Gene Symbol or |
| Probe set ID | (4 hr) | lation | Unigene ID | Name |
| 228196_s_at | 4.039798 | down | Hs.631814 | transcribed locus |
| 211307_s_at | 4.047831 | down | Hs.659872 | FCAR |
| 211363_s_at | 4.050233 | down | Hs.193268 | MTAP |
| 241666_at | 4.088215 | down | Hs.55131 | C3orf23 |
| 229676_at | 4.108218 | down | Hs.173946 | PAPD1 |
| 215043_s_at | 4.125255 | down | Hs.652536 | SMA4 |
| | | | | SMA5 |
| 211087_x_at | 4.140346 | down | Hs.588289 | MAPK14 |
| 1557918_s_at | 4.168854 | down | Hs.75231 | SLC16A1 |
| 209761_s_at | 4.178591 | down | Hs.145150 | SP110 |
| 1554767_s_at | 4.188646 | down | Hs.352671 | CRYZL1 |
| 222614_at | 4.204624 | down | Hs.34136 | RWDD2B |
| 221194_s_at | 4.217304 | down | Hs.531701 | RNFT1 |
| 1552703_s_at | 4.232619 | down | Hs.348365 | CASP1 |
| | | | | COP1 |
| 231918_s_at | 4.241984 | down | Hs.277154 | GFM2 |
| 242288_s_at | 4.257984 | down | Hs.532815 | EMILIN2 |
| 203622_s_at | 4.348037 | down | Hs.262858 | PNO1 |
| 1558015_s_at | 4.413946 | down | Hs.699451 | ACTR2 |
| 1552486_s_at | 4.455582 | down | Hs.410388 | LACTB |
| 1558775_s_at | 4.467875 | down | Hs.372000 | NSMAF |
| 210180_s_at | 4.50081 | down | Hs.533122 | SFRS10 |
| 1553530_a_at | 4.503789 | down | Hs.695946 | ITGB1 |
| 1555814_a_at | 4.505738 | down | Hs.247077 | RHOA |
| 212142_at | 4.618687 | down | Hs.460184 | MCM4 |
| 226825_s_at | 4.627178 | down | Hs.479766 | TMEM165 |
| 1553940_a_at | 4.633946 | down | Hs.578684 | LRRC28 |
| 223925_s_at | 4.737739 | down | Hs.602015 | LOC100130332 |
| | | | | LOC100134793 |
| | | | | LUZP6 |
| | | | | MTPN |
| 76897_s_at | 4.767624 | down | Hs.522351 | FKBP15 |
| 206116_s_at | 5.055958 | down | Hs.133892 | TPM1 |
| 232675_s_at | 5.136206 | down | Hs.504998 | UCKL1 |
| 204427_s_at | 5.145536 | down | Hs.75914 | TMED2 |
| 227364_at | 5.153504 | down | | |
| 220494_s_at | 5.194894 | down | | |
| 215073_s_at | 5.237517 | down | Hs.701977 | NR2F2 |
| 229835_s_at | 5.263865 | down | Hs.656865 | SLMO2 |
| 208748_s_at | 5.305004 | down | Hs.179986 | FLOT1 |
| 207495_at | 5.334993 | down | Hs.656060 | RAB28 |
| 1554424_at | 5.341551 | down | Hs.518760 | FIP1L1 |
| 207419_s_at | 5.382367 | down | Hs.517601 | RAC2 |
| 210935_s_at | 5.491971 | down | Hs.128548 | WDR1 |
| 226570_at | 5.504646 | down | Hs.477789 | ATP1B3 |
| 227299_at | 5.520281 | down | Hs.709250 | CCNI |
| 223341_s_at | 5.524223 | down | Hs.480815 | SCOC |
| 203372_s_at | 5.566165 | down | Hs.485572 | SOCS2 |
| 1554768_a_at | 5.570022 | down | Hs.591697 | MAD2L1 |
| 219858_s_at | 5.710344 | down | Hs.708489 | FLJ20160 |
| 216521_s_at | 5.722223 | down | Hs.558537 | BRCC3 |
| 205461_at | 5.779909 | down | Hs.524788 | RAB35 |
| 212009_s_at | 5.792535 | down | Hs.337295 | STIP1 |
| 201946_s_at | 5.829165 | down | Hs.189772 | CCT2 |
| 241733_at | 5.868862 | down | Hs.208701 | C18orf54 |
| 219599_at | 5.968014 | down | Hs.648394 | EIF4B |
| 229212_at | 6.051483 | down | Hs.644056 | transcribed locus |
| 233878_s_at | 6.079963 | down | Hs.255932 | XRN2 |
| 221276_s_at | 6.246901 | down | Hs.712631 | SYNC1 |
| 202226_s_at | 6.256392 | down | Hs.638121 | CRK |
| 231972_at | 6.439062 | down | Hs.656166 | CDNA: FLJ21028 |
| | | | | fis, clone |
| | | | | CAE07155 |
| 1555154_a_at | 6.482312 | down | Hs.510324 | QKI |
| 242277_at | 6.48723 | down | Hs.701344 | transcribed locus |
| 212105_s_at | 6.670136 | down | Hs.191518 | DHX9 |
| 224407_s_at | 6.680334 | down | Hs.444247 | RP6-213H19.1 |
| 227658_s_at | 6.693072 | down | Hs.41086 | PLEKHA3 |
| 215719_x_at | 6.719481 | down | Hs.244139 | FAS |
| 213562_s_at | 6.760106 | down | Hs.71465 | SQLE |
| 216252_x_at | 6.859726 | down | Hs.244139 | FAS |
| 223143_s_at | 7.051 | down | Hs.485915 | C6orf166 |
| 209535_s_at | 7.296443 | down | | |
| 217294_s_at | 7.459872 | down | Hs.517145 | ENO1 |
| 219927_at | 7.737007 | down | Hs.579828 | FCF1 |
| 244187_at | 7.950194 | down | Hs.648463 | transcribed locus |
| 231370_at | 8.033558 | down | Hs.707434 | transcribed locus |
| 204426_at | 8.122973 | down | Hs.75914 | TMED2 |
| 229787_s_at | 8.148592 | down | Hs.705676 | transcribed locus |
| 227260_at | 8.706609 | down | Hs.525163 | ANKRD10 |
| 1565717_s_at | 8.748013 | down | Hs.513522 | FUS |
| 211102_s_at | 9.204724 | down | Hs.655593 | LILRA2 |
| 243495_s_at | 9.238962 | down | Hs.712948 | MRNA; cDNA |
| | | | | DKFZp686E18224 |
| 222611_s_at | 9.446891 | down | Hs.213198 | PSPC1 |
| 229713_at | 9.591681 | down | Hs.644929 | transcribed locus |
| 238474_at | 9.751438 | down | Hs.510375 | NUP43 |
| 228746_s_at | 10.68661 | down | Hs.518265 | transcribed locus |
| 229128_s_at | 10.89904 | down | Hs.656466 | transcribed locus |
| 230659_at | 11.50984 | down | Hs.673002 | transcribed locus |
| 1555193_a_at | 11.88252 | down | Hs.655904 | ZNF277 |
| AFFX- | 17.0748 | down | | |
| HUMRGE/M1 | | | | |
| 0098_5_at | | | | |
| 230265_at | 17.40556 | down | Hs.181300 | SEL1L |
| 231576_at | 17.47066 | down | Hs.702816 | transcribed locus |
| 203032_s_at | 19.22807 | down | Hs.592490 | FH |
| 213872_at | 27.33301 | down | Hs.592644 | transcribed locus |
| 232148_at | 4.004654 | up | Hs.372000 | NSMAF |
| 243768_at | 4.01657 | up | Hs.696546 | transcribed locus |
| 243158_at | 4.025012 | up | | |
| 230998_at | 4.031707 | up | Hs.653966 | transcribed locus |
| 241893_at | 4.051749 | up | Hs.655355 | transcribed locus |
| 242837_at | 4.085199 | up | Hs.469970 | SFRS4 |
| 1559117_at | 4.101708 | up | Hs.634052 | CDNA FLJ34664 |
| | | | | fis, clone |
| | | | | LIVER2000592 |
| 244427_at | 4.101731 | up | Hs.270845 | KIF23 |
| 237388_at | 4.122815 | up | Hs.49105 | GLMN |
| 237768_x_at | 4.129501 | up | | |
| 1562511_at | 4.136996 | up | Hs.532411 | LYST |
| 229871_at | 4.157612 | up | Hs.612332 | SAMD4B |
| 230712_at | 4.158728 | up | Hs.655246 | KIAA1245 |
| | | | | LOC100132406 |
| | | | | NBPF1 |
| | | | | NBPF10 |
| | | | | NBPF11 |
| | | | | NBPF14 |
| | | | | NBPF15 |
| | | | | NBPF16 |
| | | | | NBPF20 |
| | | | | NBPF3 |
| | | | | NBPF8 |
| | | | | NBPF9 |
| | | | | RP3-377D14.1 |
| | | | | XXyac-YX155B6.1 |
| 215191_at | 4.161385 | up | Hs.636888 | CDNA FLJ14085 |
| | | | | fis, clone |
| | | | | HEMBB1002534 |
| 1556338_at | 4.162479 | up | Hs.662144 | CDNA FLJ39845 |
| | | | | fis, clone |
| | | | | SPLEN2014452 |
| 229514_at | 4.181521 | up | Hs.410231 | C14orf118 |
| 234723_x_at | 4.183379 | up | Hs.677287 | CDNA: FLJ21228 |
| | | | | fis, clone |
| | | | | COL00739 |
| 234032_at | 4.186586 | up | Hs.684536 | transcribed locus |
| 234989_at | 4.195736 | up | | TncRNA |
| 232597_x_at | 4.208668 | up | Hs.210367 | SFRS2IP |
| 230651_at | 4.216365 | up | Hs.666664 | transcribed locus |
| 230099_at | 4.217873 | up | Hs.605074 | transcribed locus |
| 243303_at | 4.220261 | up | | |
| 1558710_at | 4.230851 | up | Hs.687438 | CDNA FLJ40669 |
| | | | | fis, clone |
| | | | | THYMU2020883 |
| 244015_at | 4.26262 | up | | |
| 217164_at | 4.266195 | up | | |
| 240247_at | 4.27683 | up | | |
| 242261_at | 4.299726 | up | | |
| 242476_at | 4.309448 | up | Hs.605126 | transcribed locus |
| 1558467_a_at | 4.323054 | up | Hs.656444 | Clone |
| | | | | IMAGE: 125405, |
| | | | | mRNA sequence |
| 232094_at | 4.359978 | up | Hs.633566 | C15orf29 |
| 1560145_at | 4.360516 | up | Hs.709388 | MKLN1 |
| 239091_at | 4.379727 | up | Hs.659152 | transcribed locus |
| 229422_at | 4.417276 | up | Hs.584782 | NRD1 |
| 216211_at | 4.425798 | up | Hs.659130 | MRNA; cDNA |
| | | | | DKFZp564A023 |
| | | | | (from clone |
| | | | | DKFZp564A023) |
| 1559490_at | 4.449507 | up | Hs.518414 | LRCH3 |
| 1553349_at | 4.464808 | up | Hs.696080 | ARID2 |
| 243527_at | 4.476238 | up | | |
| 240008_at | 4.490335 | up | Hs.656290 | transcribed locus |
| 215204_at | 4.501104 | up | Hs.661961 | CDNA FLJ14090 |
| | | | | fis, clone |
| | | | | MAMMA1000264 |
| 242431_at | 4.504952 | up | | |
| 233300_at | 4.538653 | up | Hs.660696 | CDNA FU11548 |
| | | | | fis, clone |
| | | | | HEMBA1002944 |
| 234148_at | 4.56567 | up | Hs.677340 | CDNA: FLJ21585 |
| | | | | fis, clone |
| | | | | COL06903 |
| 238558_at | 4.60338 | up | Hs.674068 | transcribed locus |
| 232889_at | 4.617942 | up | | GUSBP1 |
| 1562062_at | 4.670624 | up | Hs.655246 | KIAA1245 |
| | | | | LOC100132406 |
| | | | | NBPF1 |
| | | | | NBPF10 |
| | | | | NBPF11 |
| | | | | NBPF14 |
| | | | | NBPF15 |
| | | | | NBPF16 |
| | | | | NBPF20 |
| | | | | NBPF3 |
| | | | | NBPF8 |
| | | | | NBPF9 |
| | | | | RP3-377D14.1 |
| | | | | XXyac-YX155B6.1 |
| 229483_at | 4.685478 | up | Hs.657369 | CDNA FLJ42331 |
| | | | | fis, clone |
| | | | | TSTOM2000588 |
| 221899_at | 4.689089 | up | Hs.507680 | N4BP2L2 |
| 243037_at | 4.723519 | up | | |
| 237632_at | 4.724212 | up | | |
| 244061_at | 4.799224 | up | | |
| 213700_s_at | 4.800273 | up | Hs.655868 | transcribed locus |
| 232529_at | 4.812432 | up | Hs.531587 | SP3 |
| 223494_at | 4.876228 | up | Hs.500842 | MGEA5 |
| 238735_at | 4.877998 | up | | |
| 201694_s_at | 4.88721 | up | Hs.326035 | EGR1 |
| 235023_at | 4.917645 | up | Hs.511668 | VPS13C |
| 1562194_at | 4.956551 | up | Hs.684712 | Full length inser [symbol] |
| | | | | cDNA clone |
| | | | | YW28D08 |
| 243964_at | 4.956945 | up | Hs.605805 | transcribed locus |
| 228105_at | 4.965934 | up | Hs.655980 | transcribed locus |
| 241775_at | 4.987032 | up | Hs.662113 | CDNA FLJ26437 |
| | | | | fis, clone |
| | | | | KDN02067 |
| 242233_at | 5.029235 | up | Hs.665440 | transcribed locus |
| 242558_at | 5.068929 | up | Hs.658202 | CDNA FLJ45490 |
| | | | | fis, clone |
| | | | | BRTHA2005831 |
| 235716_at | 5.075923 | up | Hs.569031 | transcribed locus |
| 1563130_a_at | 5.101688 | up | Hs.661154 | MRNA full length |
| | | | | insert cDNA clone |
| | | | | EUROIMAGE |
| | | | | 626063 |
| 242673_at | 5.143738 | up | Hs.599613 | transcribed locus |
| 227576_at | 5.161981 | up | Hs.276976 | CDNA FLJ42015 |
| | | | | fis, clone |
| | | | | SPLEN2032813 |
| 242121_at | 5.171345 | up | Hs.653288 | RNF12 |
| 239243_at | 5.176998 | up | Hs.434401 | ZNF638 |
| 239784_at | 5.187452 | up | Hs.669403 | transcribed locus |
| 206115_at | 5.250307 | up | Hs.534313 | EGR3 |
| 239937_at | 5.284644 | up | Hs.706835 | ZNF207 |
| 228723_at | 5.290023 | up | Hs.656678 | CDNA FLJ30445 |
| | | | | fis, clone |
| | | | | BRACE2009238 |
| 235757_at | 5.328636 | up | Hs.675679 | transcribed locus |
| 231281_at | 5.335745 | up | | |
| 235138_at | 5.36946 | up | Hs.662054 | transcribed locus |
| 1565703_at | 5.505036 | up | Hs.75862 | SMAD4 |
| 230292_at | 5.506033 | up | Hs.709358 | LOC100131993 |
| 230494_at | 5.543806 | up | Hs.187946 | SLC20A1 |
| 242008_at | 5.598142 | up | Hs.671336 | transcribed locus |
| 230387_at | 5.76847 | up | Hs.659630 | transcribed locus |
| 1569181_x_at | 5.881034 | up | Hs.662486 | transcribed locus |
| 215269_at | 6.028463 | up | Hs.126221 | TMEM1 |
| 1557543_at | 6.033797 | up | Hs.684024 | transcribed locus |
| 1565886_at | 6.471508 | up | Hs.621480 | Full length inser [symbol] |
| | | | | cDNA clone |
| | | | | ZB94A08 |
| 1569180_at | 6.566242 | up | Hs.662486 | transcribed locus |
| 233271_at | 6.631978 | up | Hs.677062 | CDNA FLJ11709 |
| | | | | fis, clone |
| | | | | HEMBA1005133 |
| 1559156_at | 6.635394 | up | Hs.658076 | CDNA clone |
| | | | | IMAGE: 5266106 |
| 215611_at | 6.663847 | up | Hs.511504 | TCF12 |
| 242068_at | 6.670528 | up | Hs.603603 | transcribed locus |
| 217591_at | 6.680076 | up | Hs.677805 | transcribed locus |
| 1557527_at | 6.746184 | up | Hs.675708 | CDNA FLJ33848 |
| | | | | fis, clone |
| | | | | CTONG2005567 |
| 207746_at | 6.833061 | up | Hs.241517 | POLQ |
| 235847_at | 6.946846 | up | Hs.661841 | transcribed locus |
| 243827_at | 7.25954 | up | Hs.601123 | transcribed locus |
| 239516_at | 7.304538 | up | Hs.125291 | transcribed locus |
| 241681_at | 7.460481 | up | Hs.656858 | transcribed locus |
| 239597_at | 7.573097 | up | | |
| 1556590_s_at | 7.724134 | up | Hs.633049 | CDNA FLJ25645 |
| | | | | fis, clone |
| | | | | SYN00113 |
| 236114_at | 7.796954 | up | Hs.666463 | transcribed locus |
| 203665_at | 7.974161 | up | Hs.517581 | HMOX1 |
| 235879_at | 8.350311 | up | Hs.478000 | MBNL1 |
| 243149_at | 8.513012 | up | Hs.669156 | transcribed locus |
| 230332_at | 9.280883 | up | Hs.654700 | ZCCHC7 |
| 222371_at | 9.376632 | up | Hs.675666 | transcribed locus |
| 235925_at | 9.392304 | up | Hs.511504 | TCF12 |
| 236561_at | 13.18963 | up | Hs.494622 | TGFBR1 |
| 225239_at | 16.84248 | up | Hs.593027 | CDNA FLJ26120 |
| | | | | fis, clone |
| | | | | SYN00419 |

[symbol]

indicates data missing or illegible when filed

TABLE 3
Gene ontology analysis.

| | | | | | Fold |
|---|---|---|---|---|---|
| | | | | | Enrich- |
| Term | Count | % | P-value | Genes | ment |
| BP00206: | 12 | 6.70% | 0.0019 | 244427_at | 3.057853 |
| Chromosome | | | | 1554768_a_at | |
| segregation | | | | 230292_at | |
| | | | | 206116_s_at | |
| | | | | 210935_s_at | |
| | | | | 241775_at | |
| | | | | 243303_at | |
| | | | | 1553530_a_at | |
| | | | | 1558015_s_at | |
| | | | | 1562062_at | |
| | | | | 239597_at | |
| | | | | 229514_at | |
| BP00032: | 10 | 5.59% | 0.011 | 243158_at | 2.746405 |
| Purine | | | | 242121_at | |
| metabolism | | | | 231576_at | |
| | | | | 206116_s_at | |
| | | | | 1553530_a_at | |
| | | | | 227260_at | |
| | | | | 216211_at | |
| | | | | 235757_at | |
| | | | | 211363_s_at | |
| | | | | 239597_at | |
| | | | | 242068_at | |
| BP00001: | 9 | 5.03% | 0.038 | 213700_s_at | 2.352284 |
| Carbohydrate | | | | 215043_s_at | |
| metabolism | | | | 215043_s_at | |
| | | | | 243303_at | |
| | | | | 217294_s_at | |
| | | | | 230659_at | |
| | | | | 232889_at | |
| | | | | 1554767_s_at | |
| | | | | 1563130_a_at | |
| | | | | 242261_at | |
| BP00101: | 9 | 5.03% | 0.022 | 219599_at | 2.617163 |
| Sulfur | | | | 230998_at | |
| metabolism | | | | 215719_x_at | |
| | | | | 216252_x_at | |
| | | | | 1562062_at | |
| | | | | 230712_at | |
| | | | | 206116_s_at | |
| | | | | 217294_s_at | |
| | | | | 244015_at | |
| | | | | 243149_at | |
| | | | | 1563130_a_at | |
| | | | | 242261_at | |
| | | | | 239516_at | |
| BP00281: | 8 | 4.47% | 0.022 | 232675_s_at | 2.853996 |
| Oncogenesis | | | | 237768_x_at | |
| | | | | 231576_at | |
| | | | | 206116_s_at | |
| | | | | 1565717_s_at | |
| | | | | 211307_s_at | |
| | | | | 1562062_at | |
| | | | | 215191_at | |
| BP00180: | 8 | 4.47% | 0.0098 | 206115_at | 3.376069 |
| Detoxification | | | | 215043_s_at | |
| | | | | 230265_at | |
| | | | | 242008_at | |
| | | | | 1562062_at | |
| | | | | 230712_at | |
| | | | | 1554424_at | |
| | | | | 236561_at | |
| | | | | 232529_at | |
| BP00114: | 8 | 4.47% | 0.0001 | 242121_at | 5.385946 |
| MAPKKK | | | | 229835_s_at | |
| cascade | | | | 206116_s_at | |
| | | | | 230659_at | |
| | | | | 238474_at | |
| | | | | 230651_at | |
| | | | | 239243_at | |
| | | | | 229514_at | |
| BP00005: | 7 | 3.91% | 0.016 | 213700_s_at | 3.450612 |
| Glycolysis | | | | 1556590_s_at | |
| | | | | 228105_at | |
| | | | | 228196_s_at | |
| | | | | 243303_at | |
| | | | | 217294_s_at | |
| | | | | 242288_s_at | |
| | | | | 1562062_at | |
| BP00033: | 7 | 3.91% | 0.011 | 229128_s_at | 3.773099 |
| Pyrimidine | | | | 232675_s_at | |
| metabolism | | | | 216521_s_at | |
| | | | | 235023_at | |
| | | | | 1555154_a_a | |
| | | | | 211307_s_at | |
| | | | | 1554767_s_at | |
| BP00129: | 6 | 3.35% | 0.040 | 229128_s_at | 3.184482 |
| Endocytosis | | | | 223341_s_at | |
| | | | | 230659_at | |
| | | | | 238474_at | |
| | | | | 211087_x_at | |
| | | | | 232529_at | |

While the present disclosure has been described with reference to what are presently considered to be the preferred examples, it is to be understood that the disclosure is not limited to the disclosed examples. To the contrary, the disclosure is intended to cover various modifications and equivalent arrangements included within the spirit and scope of the appended claims.

All publications, patents and patent applications are herein incorporated by reference in their entirety to the same extent as if each individual publication, patent or patent disclosure was specifically and individually indicated to be incorporated by reference in its entirety.

### FULL CITATIONS FOR REFERENCES REFERRED TO IN THE SPECIFICATION

8. Barlogie B, Tricot G, Anaissie E, et al. Thalidomide and hematopoietic-cell transplantation for multiple myeloma. N Engl J Med. 2006; 354:1021-1030.
9. van Rhee F, Dhodapkar M, Shaughnessy J D, Jr., et al. First thalidomide clinical trial in multiple myeloma: a decade. Blood. 2008; 112:1035-1038.
12. Feldmeier H, Bienzle U, Dohring E, Dietrich M. Flubendazole versus mebendazole in intestinal helminthic infections. Acta Trop. 1982; 39:185-189.
13. Lassegue A, Estavoyer J M, Minazzi H, et al. [Treatment of human alveolar echinococcosis with flubendazole. Clinical, morphological and immunological study]. Gastroenterol Clin Biol. 1984; 8:314-320.
14. Tellez-Giron E, Ramos M C, Dufour L, et al. Treatment of neurocysticercosis with flubendazole. Am J Trop Med Hyg. 1984; 33:627-631.
15. Fuchs R. Flubendazole. Vol. 31. Geneva: World Health Organization; 1993.
16. Moreno L, Alvarez L, Mottier L, Virkel G, Bruni S S, Lanusse C. Integrated pharmacological assessment of flubendazole potential for use in sheep: disposition kinetics, liver metabolism and parasite diffusion ability. J Vet Pharmacol Ther. 2004; 27:299-308.
17. Doudican N, Rodriguez A, Osman I, Orlow S J. Mebendazole induces apoptosis via Bcl-2 inactivation in chemoresistant melanoma cells. Mol Cancer Res. 2008; 6:1308-1315.
18. Sasaki J, Ramesh R, Chada S, Gomyo Y, Roth J A, Mukhopadhyay T. The anthelmintic drug mebendazole induces mitotic arrest and apoptosis by depolymerizing tubulin in non-small cell lung cancer cells. Mol Cancer Ther. 2002; 1:1201-1209.
19. Gupta K, Bishop J, Peck A, Brown J, Wilson L, Panda D. Antimitotic antifungal compound benomyl inhibits brain microtubule polymerization and dynamics and cancer cell proliferation at mitosis, by binding to a novel site in tubulin. Biochemistry. 2004; 43:6645-6655.
20. Cumino A C, Elissondo M C, Denegri G M. Flubendazole interferes with a wide spectrum of cell homeostatic mechanisms in Echinococcus granulosus protoscoleces. Parasitol Int. 2009.
21. Jasra N, Sanyal S N, Khera S. Effect of thiabendazole and fenbendazole on glucose uptake and carbohydrate metabolism in Trichuris globulosa. Vet Parasitol. 1990; 35:201-209.
22. Lacey E, Watson T R. Activity of benzimidazole carbamates against L1210 mouse leukaemia cells: correlation with in vitro tubulin polymerization assay. Biochem Pharmacol. 1985; 34:3603-3605.
23. Loganzo F, Hari M, Annable T, et al. Cells resistant to HTI-286 do not overexpress P-glycoprotein but have reduced drug accumulation and a point mutation in alpha-tubulin. Mol Cancer Ther. 2004; 3:1319-1327.
24. He L, Yang C P, Horwitz S B. Mutations in beta-tubulin map to domains involved in regulation of microtubule stability in epothilone-resistant cell lines. Mol Cancer Ther. 2001; 1:3-10.
25. Mawji I A, Simpson C D, Hurren R, et al. Critical role for Fas-associated death domain-like interleukin-1-converting enzyme-like inhibitory protein in anoikis resistance and distant tumor formation. J Natl Cancer Inst. 2007; 99:811-822.
26. Santra M, Santra S, Roberts C, Zhang R L, Chopp M. Doublecortin induces mitotic microtubule catastrophe and inhibits glioma cell invasion. J Neurochem. 2009; 108:231-245.
27. Wood T E, Dalili S, Simpson C D, et al. A novel inhibitor of glucose uptake sensitizes cells to FAS-induced cell death. Mol Cancer Ther. 2008; 7:3546-3555.
28. Beyer C F, Zhang N, Hernandez R, et al. TTI-237: a novel microtubule-active compound with in vivo antitumor activity. Cancer Res. 2008; 68:2292-2300.
29. Roychowdhury M, Sarkar N, Manna T, et al. Sulfhydryls of tubulin. A probe to detect conformational changes of tubulin. Eur J. Biochem. 2000; 267:3469-3476.
30. Zavareh R B, Lau K S, Hurren R, et al. Inhibition of the sodium/potassium ATPase impairs N-glycan expression and function. Cancer Res. 2008; 68:6688-6697.
31. Mao X, Li X, Sprangers R, et al. Clioquinol inhibits the proteasome and displays preclinical activity in leukemia and myeloma. Leukemia. 2009; 23:585-590.
32. Grover V S, Sharma A, Singh M. Role of nitric oxide in diabetes-induced attenuation of antinociceptive effect of morphine in mice. Eur J Pharmacol. 2000; 399:161-164.
33. Chou T C, Talalay P. Quantitative analysis of dose-effect relationships: the combined effects of multiple drugs or enzyme inhibitors. Adv Enzyme Regul. 1984; 22:27-55.
34. Sharmeen S. In preparation.
35. Jordan M A, Wilson L. Microtubules as a target for anticancer drugs. Nat Rev Cancer. 2004; 4:253-265.
36. Sternlicht H, Ringel I. Colchicine inhibition of microtubule assembly via copolymer formation. J Biol Chem. 1979; 254:10540-10550.
37. Manfredi J J, Parness J, Horwitz S B. Taxol binds to cellular microtubules. J Cell Biol. 1982; 94:688-696.
38. Risinger A L, Giles F J, Mooberry S L. Microtubule dynamics as a target in oncology. Cancer Treat Rev. 2009; 35:255-261.
39. Zelnak A B. Clinical pharmacology and use of microtubule-targeting agents in cancer therapy. Methods Mol Med. 2007; 137:209-234.
40. Bhattacharyya B, Wolff J. Promotion of fluorescence upon binding of colchicine to tubulin. Proc Natl Acad Sci USA. 1974; 71:2627-2631.
41. Gao J, Huo L, Sun X, et al. The tumor suppressor CYLD regulates microtubule dynamics and plays a role in cell migration. J Biol Chem. 2008; 283:8802-8809.
42. Jordan M A, Kamath K, Manna T, et al. The primary antimitotic mechanism of action of the synthetic halichondrin E7389 is suppression of microtubule growth. Mol Cancer Ther. 2005; 4:1086-1095.
43. Dumontet C, Sikic B I. Mechanisms of action of and resistance to antitubulin agents: microtubule dynamics, drug transport, and cell death. J Clin Oncol. 1999; 17:1061-1070.
44. Huang T H, Bebawy M, Tran V H, Roufogalis B D. Specific reversal of multidrug resistance to colchicine in CEMNLB(100) cells by Gynostemma pentaphyllum extract. Phytomedicine. 2007; 14:830-839.
45. Chou T C. Preclinical versus clinical drug combination studies. Leuk Lymphoma. 2008; 49:2059-2080.
46. Morris D L, Jourdan J L, Pourgholami M H. Pilot study of albendazole in patients with advanced malignancy. Effect on serum tumor markers/high incidence of neutropenia. Oncology. 2001; 61:42-46.
47. Westermann S, Weber K. Post-translational modifications regulate microtubule function. Nat Rev Mol Cell Biol. 2003; 4:938-947.
48. Ireland C M, Gull K, Gutteridge W E, Pogson C I. The interaction of benzimidazole carbamates with mammalian microtobule protein. Biochem Pharmacol. 1979; 28:2680-2682.

1. A method of treating a hematological malignancy in a subject in need thereof comprising administering to the subject an effective amount of flubendazole and/or a pharmaceutically acceptable solvate and/or prodrug thereof.

2. The method of claim 1 comprising administering to the subject an effective amount of flubendazole and/or a pharmaceutically acceptable solvate and/or prodrug thereof, and an effective amount of a vinca alkaloid and/or a pharmaceutically acceptable salt, solvate and/or prodrug thereof.

3. The method of claim 2, wherein the flubendazole and/or a pharmaceutically acceptable solvate and/or prodrug thereof is administered before, simultaneously with, or after the vinca alkaloid and/or a pharmaceutically acceptable salt, solvate and/or prodrug thereof.

4. The method of claim 2, wherein the vinca alkaloid is selected from vinblastine, vincristine, vindesine and vinolrebine and a pharmaceutically acceptable salt, solvate and/or prodrug thereof.

5. The method of claim 1, wherein the pharmaceutically acceptable salt is a sulfate salt or a tartrate salt.

6. The method of claim 1, wherein the hematological malignancy is drug resistant to a vinca alkaloid and/or overexpresses Pgp.

7. The method of claim 1, wherein the hematological malignancy is leukemia, lymphoma or myeloma.

8. The method of claim 7, wherein the leukemia is AML, ALL or CML.

9. (canceled)

10. The method of claim 1, wherein the flubendazole and/or a pharmaceutically acceptable solvate and/or prodrug thereof and/or the vinca alkaloid and/or a pharmaceutically acceptable salt, solvate and/or prodrug thereof are comprised in a single oral dosage form or separate oral dosage forms.

11. The method of claim 1, wherein the flubendazole and/or a pharmaceutically acceptable solvate and/or prodrug thereof and/or the vinca alkaloid and/or a pharmaceutically acceptable salt, solvate and/or prodrug thereof are comprised in a single intravenous dosage form or separate intravenous dosage forms.

12. A composition comprising an effective amount of flubendazole and/or a pharmaceutically acceptable solvate and/or prodrug thereof and a vinca alkaloid and/or a pharmaceutically acceptable salt, solvate and/or prodrug thereof.

13. The composition of claim 12, wherein the vinca alkaloid is selected from vincristine, vinblastine, vindesine and vinorelbine and a pharmaceutically acceptable salt, solvate and prodrug thereof.

14. The composition of claim 12, wherein the pharmaceutically acceptable salt is a sulfate salt or a tartrate salt.

15. The composition of claim 12, wherein the composition is for treating a hematological malignancy.

16.-28. (canceled)

29. A kit comprising flubendazole and/or a pharmaceutically acceptable solvate and/or prodrug thereof and optionally a vinca alkaloid and/or a pharmaceutically acceptable salt, solvate and/or prodrug thereof for treating a hematological malignancy according to the method of claim 1.

30. A kit comprising flubendazole and/or a pharmaceutically acceptable solvate and/or prodrug thereof and instructions for use in combination with a vinca alkaloid and/or a pharmaceutically acceptable salt, solvate and/or prodrug thereof for treating a hematological malignancy according to the method of claim 1.

31. A kit comprising a vinca alkaloid and/or a pharmaceutically acceptable salt, solvate and/or prodrug thereof and instructions for use in combination with flubendazole and/or a pharmaceutically acceptable solvate and/or prodrug thereof for treating a hematological malignancy according to the method of claim 1.

32. A method according to claim 1 for reducing toxicity associated with the administration of a vinca alkaloid comprising administering an effective amount of flubendazole and/or a pharmaceutically acceptable solvate and/or prodrug thereof in combination with a vinca alkaloid and/or a pharmaceutically acceptable salt, solvate and/or prodrug thereof, wherein the quantity of the vinca alkaloid and/or a pharmaceutically acceptable salt, solvate and/or prodrug thereof administered is reduced compared to a standard treatment protocol.

33. The method of claim 32 wherein the quantity of vinca alkaloid and/or a pharmaceutically acceptable salt, solvate and/or prodrug thereof is reduce by about 5%, 10%, 15%, 20%, 25%, 30% or more.

34. A method of increasing anti-tumor efficacy of a vinca alkaloid comprising administering an effective amount of flubendazole and/or a pharmaceutically acceptable solvate and/or prodrug thereof in combination with a vinca alkaloid and/or a pharmaceutically acceptable salt, solvate and/or prodrug thereof.
